(19) United States
(12) Patent Application Publication
(10) Pub. No.: US 2024/0256821 A1
(43) Pub. Date: Aug. 1, 2024

(54) Container with Wireless Communication and Electronic Reminders

(71) Applicant: Jogan Health, LLC, Henderson, NV (US)

(72) Inventors: Alexander Hambrock, Chicago, IL (US); Coleman Iverson, Minneapolis, MN (US); Paul Baird, Chicago, IL (US); Chris Sweet, Minneapolis, MN (US); Marcus Papadopoulos, Minneapolis, MN (US); Eric Pietrowicz, Minneapolis, MN (US)

(21) Appl. No.: 18/433,677

(22) Filed: Feb. 6, 2024

Related U.S. Application Data
(63) Continuation of application No. PCT/US2022/074673, filed on Aug. 8, 2022.
(60) Provisional application No. 63/230,096, filed on Aug. 6, 2021.

(51) Int. Cl. G06K 19/077; H04B 5/70

(52) U.S. Cl. CPC G06K 19/07758; H04B 5/70

(57) ABSTRACT

A smart container that includes a bottle or other container a processor, passive radio-frequency (RF) communications tag (e.g., an RFID or NFC tag) and/or Bluetooth, Wi-Fi, and/or cellular capabilities, and a (colored) light source. The container may hold liquid, food, pills, or another consumable substance. The passive RF communications tag and/or Bluetooth, Wi-Fi, and/or cellular antenna may also be contained on a smart band, a sticker, a button, and/or a keychain or dongle. The light source may include one or more light-emitting diodes (LEDs) which can be programmed to emit unique illumination patterns. The container may also include a speaker or motor to emit audio or vibrational notifications. The container provides a method of tracking consumption by a user of a substance held in a container.

### CROSS-REFERENCE TO RELATED PATENT APPLICATIONS

This application is a bypass continuation of International Application No. PCT/US2022/074673, filed Aug. 8, 2022, which in turn claims priority to U.S. Application No. 63/230,096, filed Aug. 6, 2021. Each of these applications is incorporated herein by reference in its entirety.

### BACKGROUND

Fitness trackers, such as smartwatches and fitness bracelets, are examples of devices that can monitor a user's physical activity throughout the day. These devices help users monitor their physical health and well-being while also allowing users to tailor the devices based on their preferences. Typically, a fitness tracker is connected to a user's smartphone, tablet, or another device such that the user can monitor his or her progress in an application. For example, many pieces of software designed for personal devices, such as home computers and smart devices (e.g., a smartphone, tablet, or another device), are designed to track various aspects of the user's life, such as their exercise routine, their eating habits, and their water consumption.

While these smart devices can track the wearer's physical activities to better inform the wearer of his or her activity levels, there are still not many devices that can inform the wearer on other important aspects, for example, nutrition intake or hydration levels. Additionally, in many cases such smart devices require the user to manually enter data about their behavior, a step which can be easy to forget and difficult to do. Proper hydration is essential, but some studies show that over 75% of people have poor water consumption habits and fewer than 5% regularly consume enough water. Encouraging proper hydration can improve health and quality of life and promote healthy habits. Additionally, monitoring food intake or pill intake can help a user follow and track his or her nutritional or medical guidelines. Further, well-designed and well-built containers are more sustainable than single use containers.

### SUMMARY

Embodiments of the present technology include an assembly including a container to hold a consumable substance and a passive radio-frequency (RF) communication tag with a unique identifier. The passive RF communication tag is affixed to the container and configured to respond to a query from a smart device. The unique identifier causes the smart device to update a record to indicate that a user has consumed at least a portion of the consumable substance. The passive RF communication tag comprises at least one of a radio-frequency identification (RFID) tag or a near-field communication (NFC) tag. The unique identifier corresponds to an account of the user, is assigned to the container, and represents consumption of a predefined amount of the consumable substance by the user.

The assembly may further include a puck, a light source, and a power supply. The puck is removably coupled to the container and holds the passive RF communication tag. The light source is disposed in the puck and emits light indicating at least one of an update to the record or a prompt to the user to consume at least a portion of the contents of the container. The power supply is disposed in the puck and operably coupled to the light source, to power the light source.

The assembly may further include a band or a dongle. The band is disposed about the container to hold the passive RF communication tag. The dongle is removably coupled to the container to hold the passive RF communication tag.

The light source is coupled to the container and emits light indicating at least one of an update to the record, a prompt to the user to consume at least a portion of the contents of the container, or an interrogation of the passive RF communication tag.

Another embodiment includes a tap band including a band, a passive radio-frequency (RF) communication tag, and a light source. The band is sized to fit about a container holding a consumable substance and/or about a wrist of a person consuming the consumable substance from the container. The passive RF communication tag is equipped with a unique identifier, affixed to the band, and configured to respond to a query from a smart device. The unique identifier causes the smart device to update a record to indicate that the person has consumed at least a portion of the consumable substance from the container. The light source is disposed on the band and emits light indicating at least one of an update to the record or a prompt to the person to consume the consumable substance from the container. The passive RF communication tag comprises at least one of a radio-frequency identification (RFID) tag or a near-field communication (NFC) tag.

The tap band may further include a light pipe. The light pipe is removably attached to the band and in optical communication with the light source behind a fabric housing. The light pipe diffuses the light from the light source.

In one embodiment the tap band may be removably affixed to the container. In another embodiment the tap band is removably affixed to the wrist of the person.

The tap band may further include a speaker or an actuator. The speaker is affixed to and/or inside the band and configured to emit unique sound notifications. The actuator affixed to and/or inside the band and configured to emit unique vibration patterns.

Yet another embodiment includes a passive radio-frequency (RF) communication tag configured to be affixed to at least one of a container holding an consumable substance or a wrist of a person and store a unique identifier. The unique identifier is configured to respond to a query from a smart device and causes the smart device to update a record to indicate that the person has consumed at least a portion of a consumable substance from the container. The passive RF communication tag comprises at least one of a radio-frequency identification (RFID) tag or a near-field communication (NFC) tag. The unique identifier corresponds to an account for the person, is assigned to the container, and represents consumption of a predefined amount of the consumable substance by the person.

Yet another embodiment includes a method of tracking consumption of a consumable substance stored in a container with a passive radio-frequency (RF) communication tag affixed thereto. This method includes consuming, by a person, at least a portion of the ingestible substance stored in the container and interrogating the passive RF communication tag with a query from a smart device. In response to the interrogation the passive RF communication tag responds to the query with a unique identifier. The smart device detects the unique identifier. The smart device updates a record indicating that the person has consumed at least a portion of the contents of the container.

The updating of the record includes adding at least of one a time or a location of the interrogation of the passive RF communication tag by the smart device to the record. The updating of the record may also include identifying a type and size of the container based on the unique identifier. The updating of the record may further include adding an amount of the portion of the contents of the container consumed by the person based on the unique identifier.

This method includes adjusting, by the person, the amount of the portion of the consumable substance consumed by the person in the record.

This method includes in response to detecting the unique identifier and before updating the record, downloading, by the smart device, an application on the smart device and associating the unique identifier with the person, the container, and an amount of the contents of the container consumed by the person for each interrogation of the passive RF communication tag.

All combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. Terminology explicitly employed herein that also may appear in any disclosure incorporated by reference should be accorded a meaning most consistent with the particular concepts disclosed herein.

### BRIEF DESCRIPTIONS OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally and/or structurally similar elements).

FIG. 1A shows a front view of a smart container with a passive radio-frequency (RF) communications tag in a smart base and a chug lid.

FIG. 1B shows a perspective side view of the smart container of FIG. 1A.

FIG. 1C shows a front view of a container portion of the smart container of FIG. 1A.

FIG. 1D shows a cross-sectional view of the attachment of the smart base to the container portion and seal of the smart container.

FIG. 1E shows a cross-sectional view of a smart base screwed onto the container portion of the smart container.

FIG. 1F shows a cross-section view of the passive RF communications tag in the holder on the smart base screwed onto the container portion of the smart container.

FIG. 2A shows a perspective view of the smart base disconnected from the rest of the smart container.

FIG. 2B shows an underside view of the smart base and the button interface to control the notifications.

FIG. 2C shows a perspective view inside of the top of the detached smart base.

FIG. 2D shows an exploded view of the smart base.

FIG. 3A shows a smart container with a straw lid.

FIG. 3B shows an exploded view of the straw lid of FIG. 3A.

FIG. 3C shows a cross-sectional view of the straw lid of FIGS. 3A and 3B.

FIG. 4A shows a smart container with a chug lid.

FIG. 4B shows an exploded view of the chug lid of FIG. 4A.

FIG. 4C shows a cross-sectional view of the chug lid of FIGS. 4A and 4B.

FIG. 5A shows a front view of a container with a smart band.

FIG. 5B shows a front view of a container with an illuminated smart band.

FIG. 5C shows an image of an illuminated smart band on a user's wrist.

FIG. 6A shows a top perspective view of an illuminated smart band.

FIG. 6B shows an underside view of the flexible light band and processor.

FIG. 6C shows a top view of the illuminated flexible light band and processor FIG. 6D shows a side view of the outside of the illuminated smart band.

FIG. 6E shows a top view of the illuminated smart band and silicone grip.

FIG. 7A shows a front view of a container with a smart band and passive RF communications tag.

FIG. 7B shows a front view of a container with an illuminated smart band and passive RF communications tag.

FIG. 8 shows a front view of a container with a passive RF communications tag sticker.

FIG. 9 shows a side view of a container with a keychain equipped with a passive RF communications tag.

FIG. 10A shows a front view of a smart button.

FIG. 10B shows a front view of a smart button on a keychain.

FIG. 10C shows a front view of a smart button with a band on a pill container.

FIG. 10D shows a front view of an illuminated smart button with a band on a pill container.

FIG. 10E shows a front view of a smart button on a keychain attached to a lunchbox.

FIG. 11A shows a back view of the smart button and battery cover.

FIG. 11B shows a diagram of the inside of a smart button.

FIG. 12 shows a bottom view of a smart button contained within a smart base and attached to a container.

### DETAILED DESCRIPTION

The present disclosure describes embodiments of a smart container that can communicate with a smartphone or other device using a short-range wireless communications protocol, such the radio-frequency identification (RFID) protocol or near-field communication (NFC) protocol. The smart container includes a container, such as a water bottle, with a removable lid and a removable base. The removable base contains or holds a passive wireless communications tag, such as a passive RFID tag or an NFC tag, that can be interrogated using a smartphone, tag reader, or other suitable device. The removable base also includes a processor, memory, timing clock, and power supply as well as light(s), speaker(s), and/or actuator(s) that can be used to remind the person using the smart container to ingest at least a portion of the contents of the smart container.

The passive wireless communications tag interfaces with software running on an external device (e.g., an app on a user's smartphone). The wireless communications tag is loaded with a uniform resource identifier (URI) payload, which is a unique sequence of characters associated with the removable smart base, with a link that passively loads onto a smartphone or other smart device. Once the smartphone has received the URI payload, it may prompt the user to download an app, register the container, or log an instance of consumption based on the previously loaded settings for the smart container. For example, the passive wireless communications tag may communicate characteristics of the smart container, such as its capacity or intended contents, to the smartphone app when the smartphone and passive wireless communications tag are brought in close proximity (e.g., within 5 cm) to each other. The user can also tap their smartphone against the passive RF communications tag when they have consumed the contents of the container, causing the app to update the user's consumption records. The passive RF communications tag pairs with software running on an external device (e.g., an app on a user's smartphone) and allows the user to receive reminders prompting the user consume a substance from the smart container. The removable smart base may also be installed on different smart containers by re-assigning the URI to represent the characteristics of the new container, e.g., via the app on the user's smartphone.

The passive wireless communications tag may be permanently affixed to the smart base or held in a separate sleeve or housing that is integrally formed with or coupled to the smart base. It can include a microchip electrically coupled to an inductive coil or antenna. The microchip includes a memory, transmitter, and receiver and, with the coil or antenna, is affixed to an insulating backing, such as a piece of plastic or paper or a ferrite backing. Incident RF waves from a smartphone or other tag reader charge the coil or antenna, powering the microchip. The microchip responds to any incident queries or commands by selectively backscattering the incident RF waves to the smartphone or tag reader according to the instructions and/or data stored in the memory.

The passive wireless communications tag can be connected to the processor in the smart base so that it can transmit information from the smartphone app to the processor, such as settings for the reminders or the tag, and/or transmit information from the processor to the smartphone app, such as device settings and health. In these cases, the smart container may also include a measurement device, such as a flow meter in the lid or a load cell in the base, that measures the volume or mass of the container contents. The processor can transmit these measurements to an external device, such as the user's smartphone, via the passive RF communications tag. For instance, a load cell in the base may be operably coupled to the passive RF communications tag and can record changes in the mass of the container contents for transmission to the user's smart device via the passive RF communications tag.

In another embodiment, the smart container may also have Bluetooth, Wi-Fi, and/or cellular capabilities in the form of an antenna and suitable transmit and receive circuitry. The user can connect the smart container to a smartphone, tablet, or other device through a Bluetooth, Wi-Fi, and/or cellular connection. The processor can transmit measurements, such as the volume of the container or the settings of the container, to an external device, such as the user's smartphone, via the Bluetooth, Wi-Fi, and/or cellular antenna. The cellular connection may be through a cellular hub or a cellular data plan. The Bluetooth, Wi-Fi, and/or cellular antenna may allow tracking of the user's preferences, the smart container's location, and/or to exchange information between the smart container and other devices.

As mentioned above, the processor, electronics, power supply (e.g., a rechargeable or replaceable battery), and light(s) for the smart container are housed in a small removable base, also called a smart base, that mechanically attaches to the container, turning the container into a smart container. The smart base is interchangeable between smart containers and can be removed from the container, e.g., for cleaning the container or replacing the battery. The smart base may have an outer silicone membrane that creates a seal when the smart base is properly secured (e.g., screwed on) to the base of a container. The seal may be water-tight or water-resistant and/or air-tight or air-permeable.

The smart base includes one or more colored lights can be arranged in any direction. For example, the one or more colored lights may be arranged facing radially outward in the smart base to produce smooth and uniform illumination from the bottom of the smart container. Alternatively, the one or more colored lights may be facing upward to illuminate the smart container body or facing downward. Light from the colored lights, which may include one or more LEDs, may diffuse through the housing to provide uniform illumination when the smart base is assembled and coupled to the container. Multi-colored lights allow for a greater variety of messages and glow sequences to be conveyed to the user. For example, the lights may notify the user of the battery level of the smart container or remind the user that it is time to consume at least a portion of the contents of the smart container.

The processor in the smart base actuates and manages power consumption by the light(s), speaker(s), and/or actuator(s). The processor controls battery draw, increasing power efficiency. The processor may also manage the intensity, pulse rate, and/or color(s) of pulses emitted by the lights, since some colors may appear brighter than others, to provide desired glow sequences. Similarly, if the smart base includes a speaker or actuator, the processor may control the sounds emits emitted by the speaker or vibrations caused by the actuator.

The messages conveyed to the user by the light(s), speaker(s), and/or actuator(s) can be simple or sophisticated, customizable, or predefined, and can indicate information about contents or status of the smart container. When the container is a smart water bottle, the messages can indicate the user's progress toward a hydration goal or the smart water bottle status. When the container is a smart pill container, the messages can prompt the user to take a pill from the container or notify the user that a pill was taken from the container. When the container is a smart food container, the smart container can indicate information to the user regarding the consumption of the contents in the container or if the contents have expired.

Inventive smart containers have a number of advantages compared to existing smart water bottles. For instance, because the electronics of the smart container are housed in a separate smart base, the user can disassemble and clean the smart container without damaging the electronics. In particular, when the smart container is equipped with a passive RF communications tag, the tag is contained within the separate smart base enabling the smart container to be washed without damaging the tag. Additionally, because the smart base may be interchangeable between smart containers, the user can use one smart base with a variety of containers based on user preference for container. The smart base can also be disassembled, allowing for the internal components to be re-used, repaired, or replaced, reducing waste. The smart base may also be reconfigurable to allow for use between multiple smart containers. Similarly, when the smart container is equipped with a Bluetooth, Wi-Fi, and/or cellular antenna, the antenna may also be contained within the separate smart base enabling the smart container to be washed without damaging the antenna.

Another advantage of the inventive smart container is the message sender or receiver's ability to program the messages conveyed by the smart water bottle through the use of multi-colored lights, audio, and/or vibrations. For example, the multi-colored lights can be used to indicate that the user should drink from the smart water bottle. Alternatively, the multi-colored lights could indicate what pill the user should take (e.g., blue light for a blue pill or red light for a red pill). The passive RF communications tag may enable the user to send and receive custom messages from the user's phone, for example. The smart container can also convey messages from non-users, for example, a message from a health care professional prompting the user to take a pill from the smart container. Similarly, when the smart container is equipped with a Bluetooth, Wi-Fi, and/or cellular antenna, the antenna may enable the user to send and receive custom messages from the user's phone. The Bluetooth, Wi-Fi, and/or cellular antenna may also allow the smart container to convey messages from non-users, for example a health care professional prompting the user to take a pill from the smart container.

### A Fully Assembled Smart Container

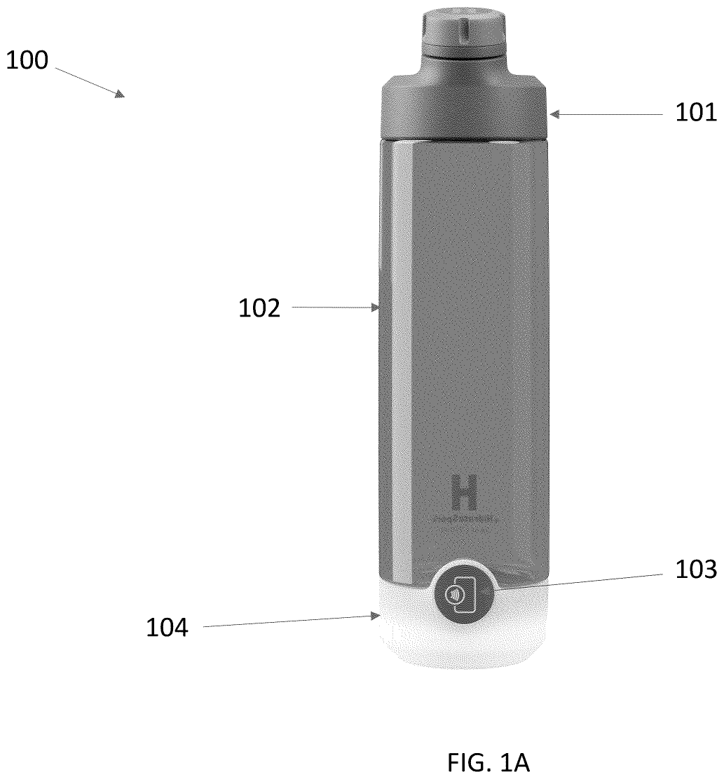

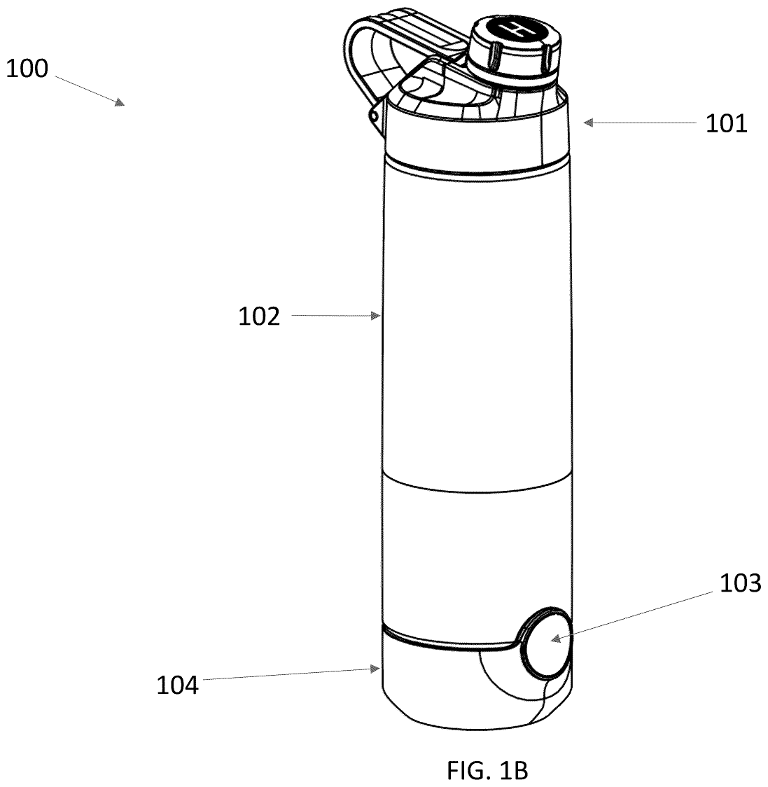

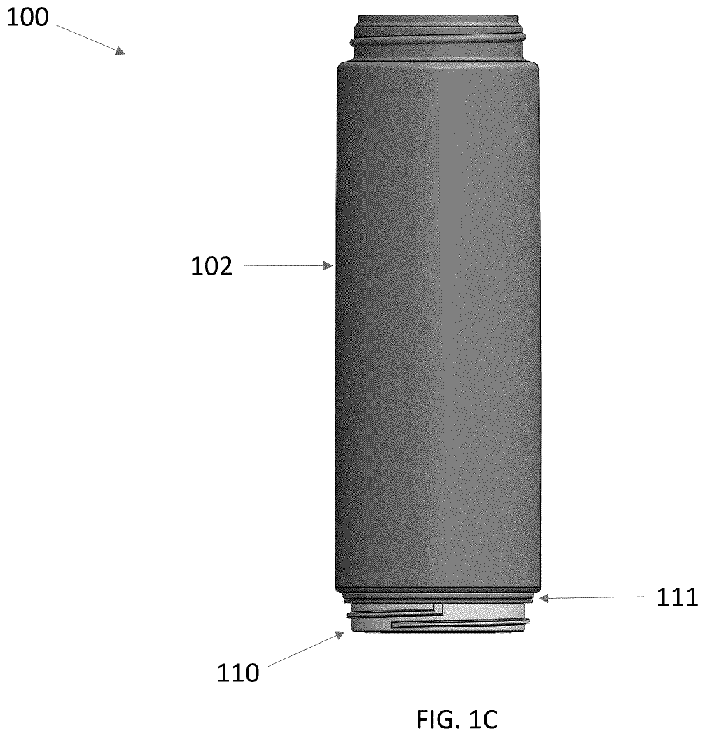

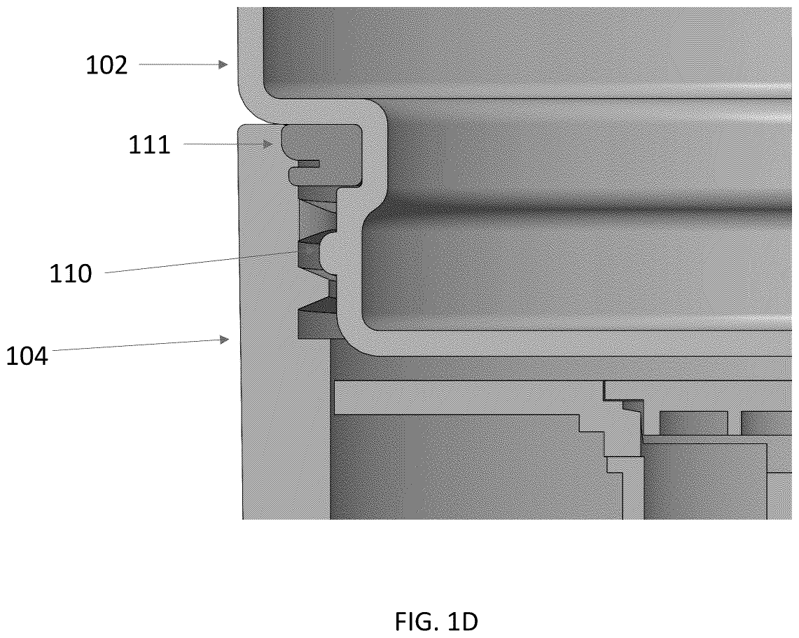

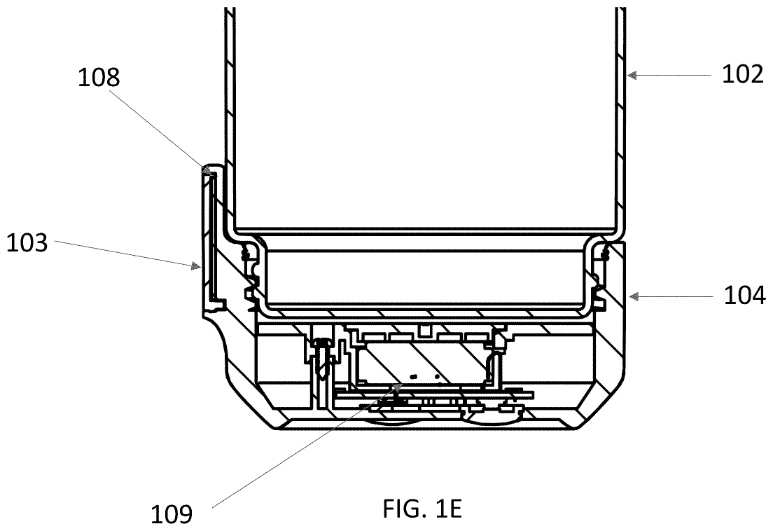

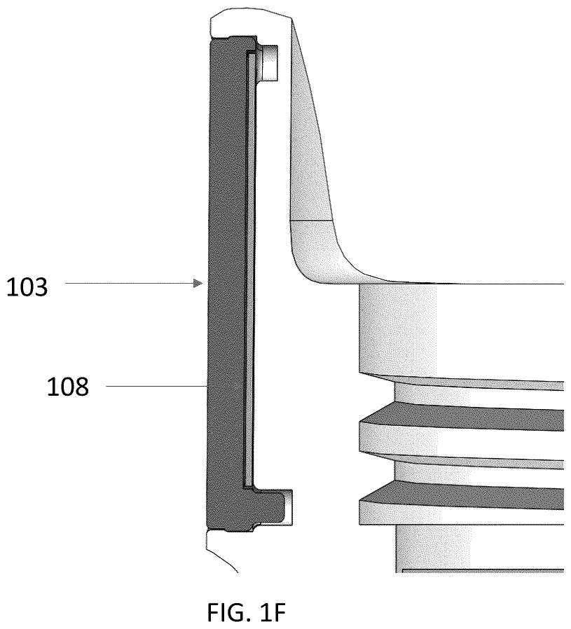

FIGS. 1A-IF show a smart container 100 equipped for wireless communication. FIGS. 1A and 1B show perspective and plan views, respectively, of the fully assembled smart container 100, which includes a bottle or container 102 with a removable lid 101 and a removable smart base 104. The removable lid 101 screws, snaps, or otherwise secures to the top of the bottle 102, which can be threaded or have a lip or other features for holding clips or snaps in place. If the lid 101 is a screw lid, it will stop when completely screwed to the container 102. Similarly, the removable smart base 104 screws, snaps, or otherwise secures to the bottom of the bottle 102, which be threaded or have a lip or other features for holding clips in place. When the smart base 104 screws to the bottom of the container 102, it has a stopping point based on the threads. When the smart base 104 is connected to the container 102 the radio frequency communication holder 103 is aligned top to bottom with the lid 101 as shown in FIG. 1A. This ensures the passive RF communications tag 108 can be easily accessed by the user and that any buttons on the smart base 104 are properly orientated to be read by the user. The removable smart base 104 includes a sleeve, pocket, or holder 103 that holds a passive RF communications tag (e.g., a passive RFID or NFC tag) 108. This passive RF communications tag 103 transforms the bottle 102 into a smart container 100 that can communicate wirelessly with an external device, such as a smartphone, for tracking a person's consumption of the contents of the bottle 102.

FIG. 1C shows the container 102 uncoupled from the smart base 104. The smart base 104 may be attached to a container via an alignment ring 110 and may be sealed to the container 102 with a seal 111 made of silicone or another suitable material. The container 102 itself may be of any material, size, or shape suitable for holding a consumable substance. For example, the container 102 may be a water bottle, a tumbler, a pill container, a food container, a collapsible container (e.g., a collapsible food or drink container or a backpack), or another suitable container for holding a consumable substance. The container 102 may be plastic, glass, metal, silicone, and/or another suitable material. The container 102 may be a variety of sizes including, but not limited to, 2, 5, 8, 10, 12, 14, 16, 17, 18, 20, 21, 22, 24, 28, 32, 40, 64, or 128 ounces.

FIGS. 1D-IF show the interface between the container 102 and the smart base 104 in greater detail. FIG. 1D shows a cross-sectional view of the container 102, the smart base 104, the alignment ring 110, and the seal 111 once the smart base 104 has been secured to (e.g., screwed onto) the container 102. FIG. 1E shows a cross-sectional view of the container 102, the smart base 104, and radio frequency communication holder 103. The holder 103 contains the passive RF communications tag 108 pinned behind a protective cover. FIG. 1F shows a cross-sectional view of the radio frequency communication holder 103 and passive RF communications tag 108. The radio frequency communication (e.g., an RFID protocol or NFC protocol) 108 is pinned inside the holder 103 between two pieces of plastic to cover and protect the passive RF communications tag 108 so that its surface is flat and separated from the air (and the container 102) by a narrow wall of plastic. The passive RF communications tag 108 is installed with adhesive on both sides to keep the passive RF communications tag 108 installed behind the holder 103. The holder 103 also insulates it the passive RF communications tag 108 from accidental damage or water. The passive RF communications tag 108 also has a ferrite backing so that the passive RF communications tag 108 works with all types of containers (e.g., plastic, glass, and metal). The ferrite backing is installed behind the passive RF communications tag 108 so that the ferrite backing is between the container 102 and the passive RF communications tag 108. The smart base 104 houses the electronics, such as a microcontroller (MCU) or other processor, a battery, additional buttons 105, 106, 107, and LEDs. The smart base 104 may also include a speaker and/or a vibrating motor or other actuator.

The container 102 may be a water bottle, a tumbler, a pill container, a food container, a collapsible consider (e.g., a collapsible food or drink container or a backpack), or another suitable container for holding a consumable substance. When the container 102 is a water bottle, the passive RF communications tag 108 may cause an application on the user's smart device that tracks the user's water consumption to log an amount of water drunk equal to the volume of the container when the user taps their smart device against the radio frequency communication holder 103 on the container 102. When the container 102 is a food container, the passive RF communications tag 108 may cause an application on the user's smart device that tracks the user's eating and nutrition to create an entry for consuming food of the type the container is specified to hold. When the container 102 is a pill container the passive RF communications tag 108 may cause an application on the user's smart device designed to monitor medication intake to create an entry for consuming the medication of the type that the container is specified to hold.

If the container 102 holds different items at the same time the application on the user's smart device might prompt the user to select from a list of possible contents. For example, the application on the user's smart device could be configured to “log substance X until told otherwise” and changed to “log substance Y” in the application after the contents are manually changed.

### Removable Smart Bases

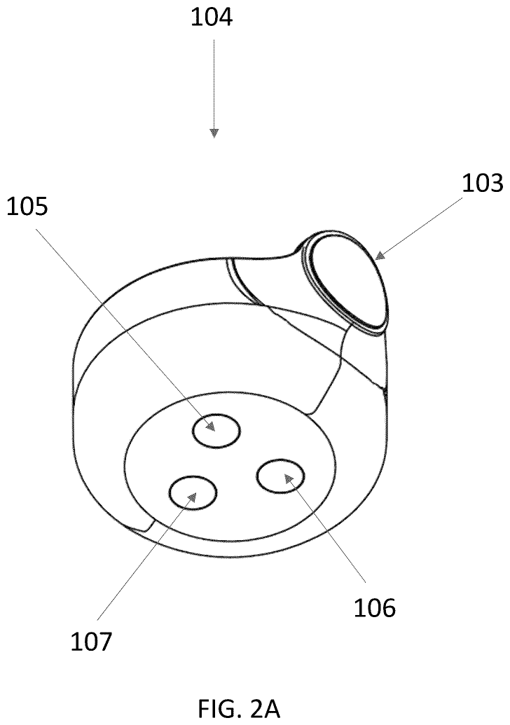

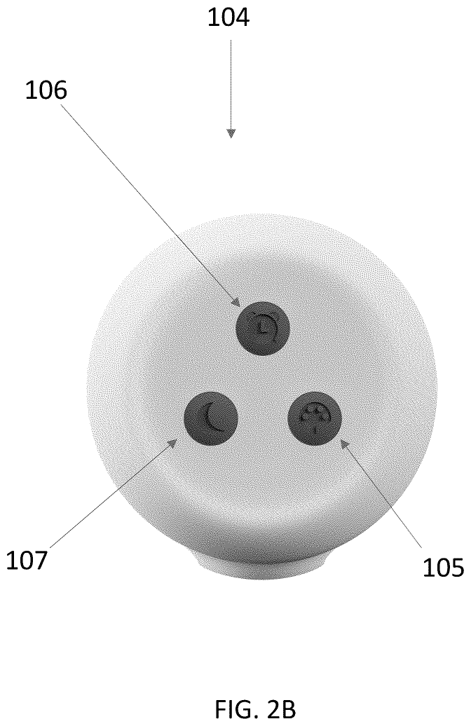

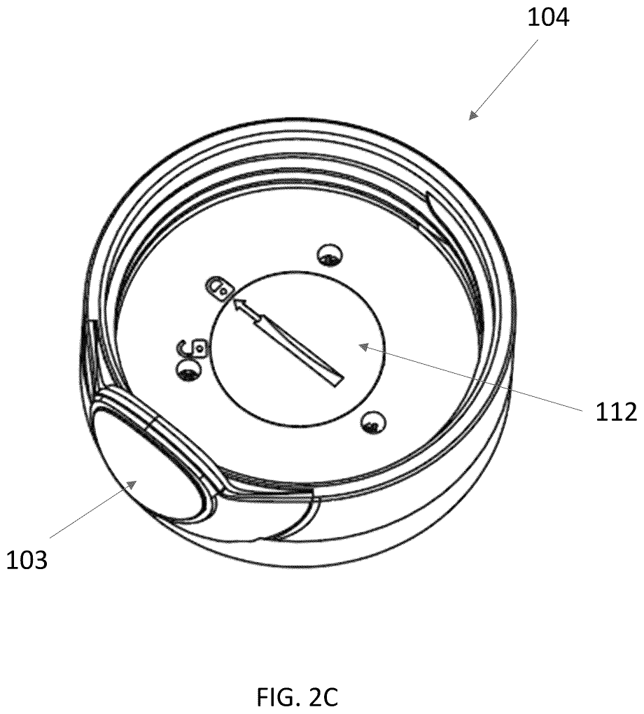

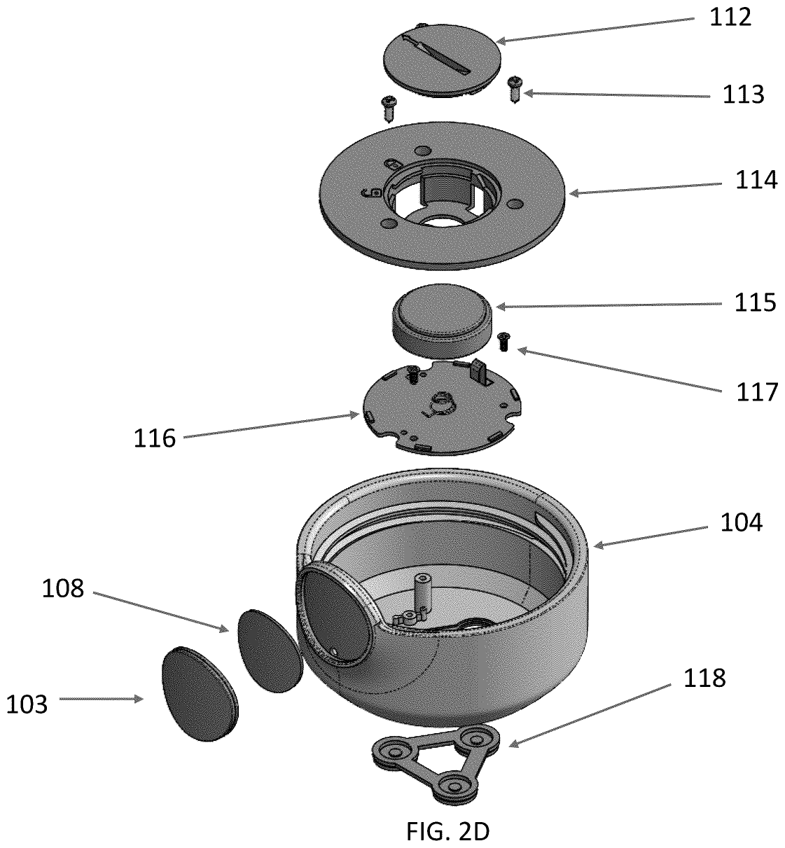

FIGS. 2A-2D show views of the smart base 104 decoupled from the container 102. FIG. 2A shows a side view of the smart base 104 and radio frequency communication holder 103 decoupled from the container 102. FIG. 2B shows a view of the underside of the smart base 104. The smart base 104 may have a color button 105, timer button 106, and/or snooze button 107 on the bottom of the smart base 104 as seen in FIGS. 2A and 2B. The buttons 105, 106, 107 may be attached to the smart base 104 as an overmold. The overmold seals the smart base 104 and protects the electronics. These buttons 105, 106, 107 may be labeled either with symbols (as shown in FIG. 2B) or words. The smart base 104 may also have LEDs inside as discussed above. The smart base 104 serves as the housing for the electronics and also acts as a diffuser to diffuse the colored light from one or more LEDs over an area viewed by the user. The smart base 104 may be made out of a translucent polypropylene. FIG. 2C shows the inside top of the detached smart base 104 and battery cover 112. FIG. 2D shows an exploded view of the smart base 104 with the battery cover 112, screws 113 to attach the battery cover 112 to the battery base 114, a battery 115, a microcontroller (MCU) or other processor 116, screws for the processor 117, the smart base 104, and the smart base button overmold 118. The LEDs may be evenly spaced around the processor 116. The only active electronics in the smart base 104 may be the LEDs and processor 116. For example, the smart base 104 may not include a sensor or load cell to measure the weight of the contents in the container 102. In this embodiment, the processor 116 and passive RF communications tag 108 may not communicate directly.

### Button Operation

The user may use the buttons 105, 106, 107 to control or modify illumination reminders. The container may provide reminders prompting the user to consume an item from the container or drink from the container through illumination or audio. The user may also interact to adjust those reminders based on the buttons 105, 106, 107 at the bottom of the smart base 104.

For instance, the buttons may include a color button 105, a timer button 106, a snooze button 107, a vibrate button (not shown), and/or a speaker button (not shown). The buttons and their operations can be hard-coded or user-adjustable, e.g., via the app. The timer button 106 can be used to initialize a timed cycle. A “Long Press” (e.g., >2 seconds) of the timer button initializes the timed cycle. The timed cycle includes at least two phases. During the first phase (the “Day Phase”) the bottle provides a reminder at a set interval (e.g., every hour) for a set period of time (e.g., 12 hours). The reminder may be visual (e.g., illumination), audio (e.g., a speaking prompting the user to consume a substance from the container), and/or sensory (e.g., vibrations). For example, while the timed cycle is running the LEDs may illuminate every hour for 12 hours. At any point during the first phase, the user may press any of the three buttons (Timer 106, Color 105, Snooze 107) while a reminder (glow, vibration, or audio cue) is in progress to interrupt the reminder and terminate it early. This reminder interrupt has no effect on future reminders, which may continue as scheduled. During the second phase (the “Night” phase) the bottle does not provide reminders at all (to account for, for example, the user being asleep). The timed cycle continues indefinitely unless put to sleep.

The bottle can be put to sleep with a Long Press of the timer button 106 while in timed cycle mode. Once the bottle is asleep, a new timed cycle can be started with a new Long Press of the timer button 106, which immediately begins a Day Phase. During the Day Phase of a timed cycle, the timer button 106 can be pressed with a Short Press (e.g., <2 seconds) to trigger a brief confirmation illumination, confirming to the user that the Timed Cycle is running.

The schedule of illuminations can be paused by pressing the snooze button 107. During the Day Phase of a timed cycle, the snooze button 107 can be pressed to set a Snooze Timer. A Snooze Timer causes the next scheduled reminder to be skipped. After the next reminder is skipped the timed cycle resumes on the normal reminder schedule. If the snooze button 107 is pressed while a Snooze Timer is active, nothing additional happens.

The user can view and select color options for the reminders using the color button 105. The color button 105 can be used at any time, whether in a Timed Cycle or Snooze cycle. Once the color button 105 is pressed, the LEDs will iterate through a looped list of possible color options for the illumination reminder. Each time the color button is pressed, the color setting changes to the next available color. Every time the selected color is changed, the LEDs briefly illuminate to demonstrate and confirm the new color. The glow color may be selected from blue, purple, red, orange, yellow, green, teal or additional colors or patterns.

### Smart Container Lids

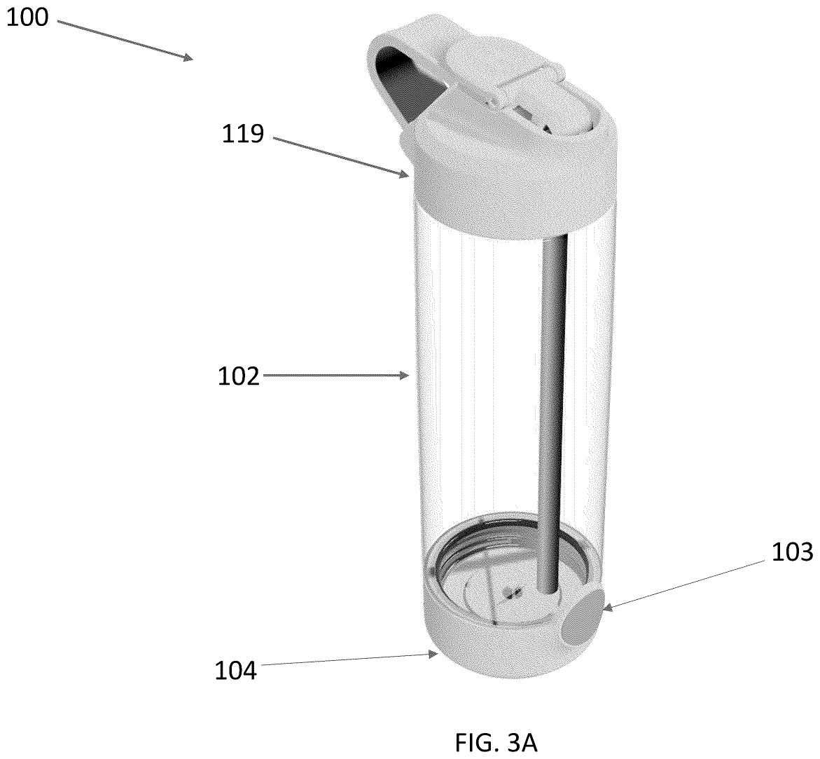

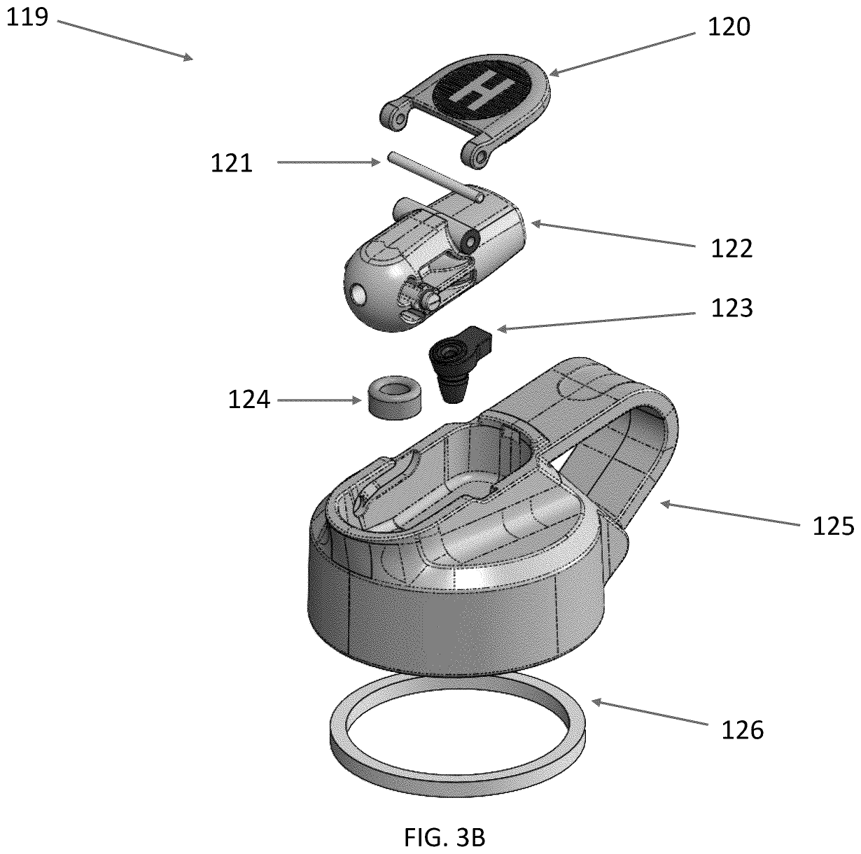

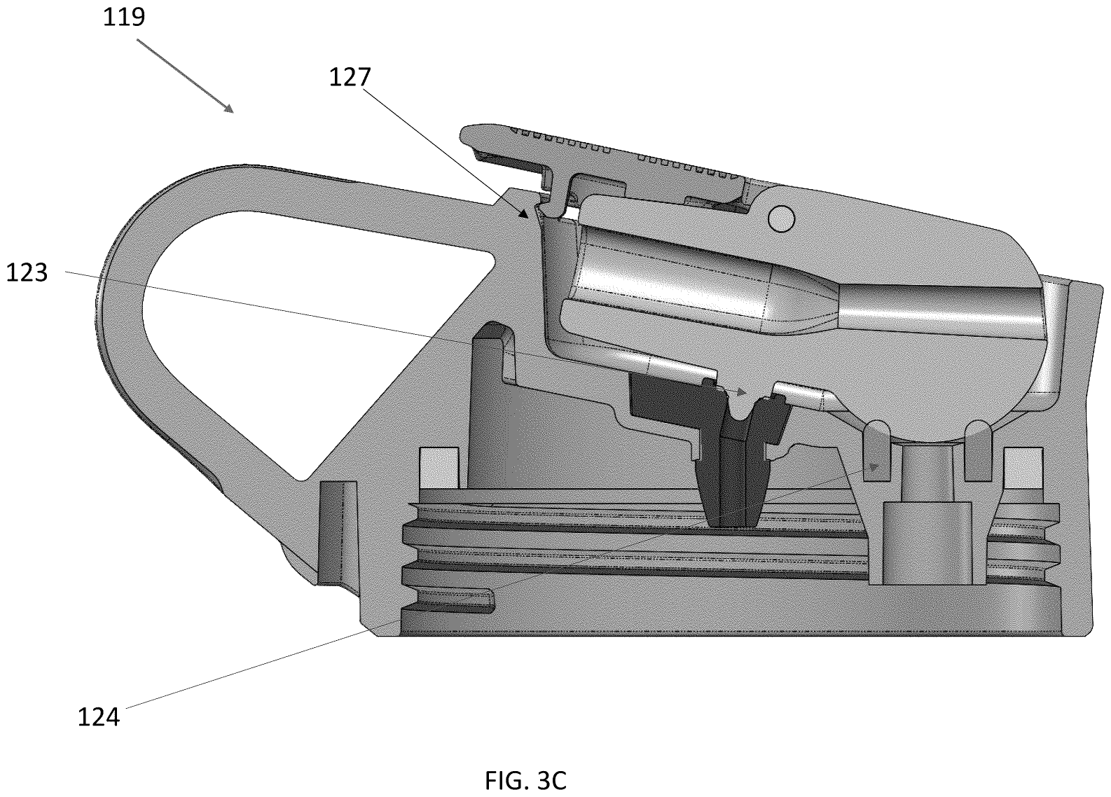

FIGS. 3A-3C show views of a straw lid 119 for use with a container 100 equipped for wireless communication. FIG. 3A shows a container 100 with a straw lid 119. FIG. 3B shows an exploded view of a straw lid 119. The straw lid 119 contains a straw spout cover 120, straw pin 121, straw spout 122, straw vent seal 123, straw spout seal 124, a lid 125, and a main lid seal 126. The straw vent seal 123, straw spout seal 124, main lid seal 126 may be made of silicone or another suitable material. FIG. 3C shows a cross-section view of the straw lid 119. The lid 125 is attached to the straw spout 122 through the straw spout cover snap 127. The straw lid 119 may also have a carry loop.

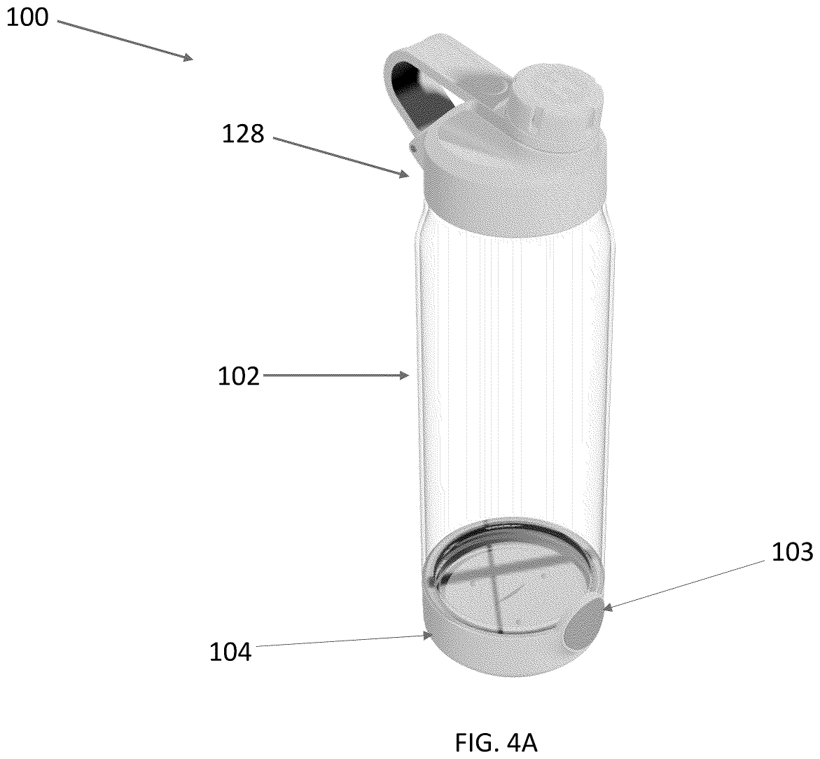

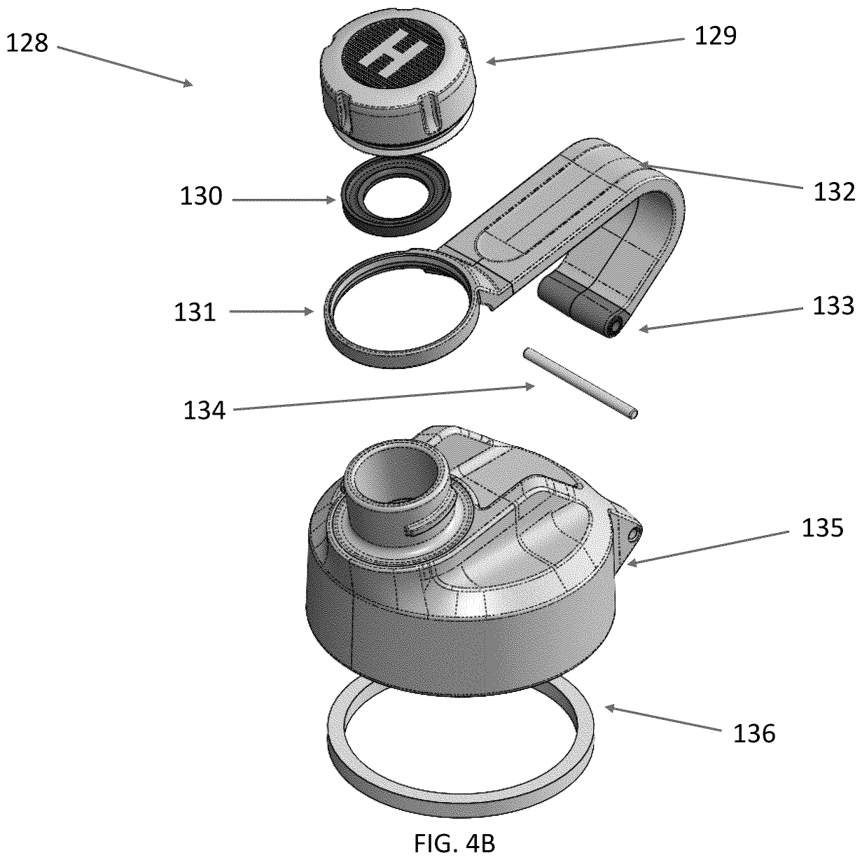

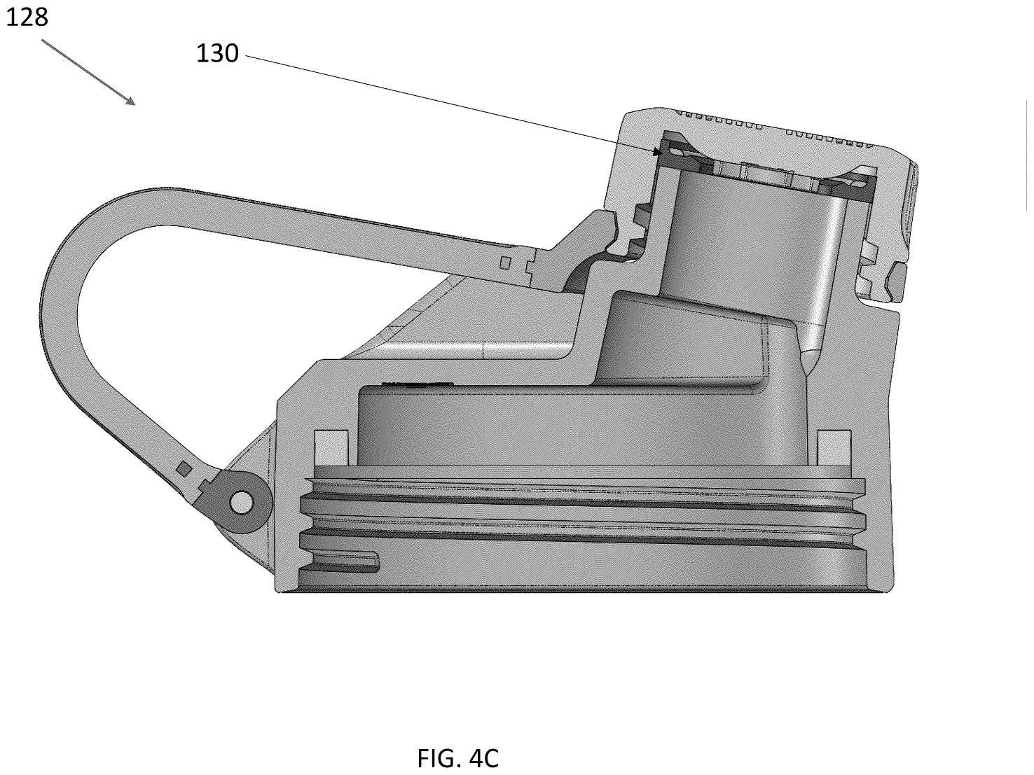

FIGS. 4A-4C show views of a chug lid 128 for use with a container 100 equipped for wireless communication. FIG. 4A shows a container 100 with a chug lid 128. FIG. 4B shows an exploded view of a chug lid 128. The chug lid 128 contains a chug cap 129, a chug cap seal 130, a chug tether ring 131, a chug tether 132, a chug tether axel 133, a chug pin 134, a lid 135, and a main lid seal 136. The chug cap seal 130 and mail lid seal 136 may be made of silicone or another suitable material. FIG. 4C shows a cross-section view of the chug lid 128. The chug lid 128 may also have a carry loop.

Passive RF Communications with the Smart Container

The passive RF communications tag 108 may use RFID or NFC technology to communicate with a nearby smart device or dedicated RFID or NFC reader. This passive RF communications tag 108 has a payload containing details, such as a unique alphanumeric identifier. The unique alphanumeric identifier represents a specific function chosen by the user and programmed in the application on the user's smart device when the smart container 100 is configured (e.g., the amount of substance consumed). Each user account may be assigned different unique alphanumeric identifiers to represent different smart containers.

The passive RF communications tag 108 may also contain codes (e.g., numbers) that map to a table of possible selections inside the software contained in an application on the user's smart device, such as the size or contents of the container 102. For example, “101=24 oz bottle” and “102=32 oz bottle.” Alternatively, the passive RF communications tag 108 may contain just the identifier and the user may map the specific passive RF communications tag 108 to a specific container 100, for example, “Tag 12345 is on a 24 oz bottle.” The passive RF communications tag 108 functions as a wireless label and may be used to initiate various actions in software installed on the user's smart device such as a smartphone, tablet, or another smart device. For example, it may initiate logging the container's 100 capacity as an amount consumed (e.g., adding 24 oz of water when the container 102 is a water bottle tracking water consumption). It may also log a single instance of an assumed event or consumption (e.g., taking one dose of medication when the container 102 is a pill container and holds several doses or pills). The container 100 may also have multiple tags 108 to record different amounts (e.g., a whole bottle, a half bottle).

As noted above, the holder 103 on the front of the smart container 100 contains a passive RF communications tag 108 behind a protective cover. When the passive RF communications tag 108 is brought in proximity to (e.g., within 5 cm of) a device running appropriate software (e.g., an application on the user's smart device), the software compares the unique alphanumeric identifier of the passive RF communications tag 108 against its database for any devices with the same identifier that belong to the active user account in the software. If no such device exists for that user account, the software prompts the user to select the size (and in some cases other properties, such as the intended use or contents of the container) of the smart container 100 associated with the passive RF communications tag 108. The software creates a new database entry specifying that container, its unique alphanumeric identifier, its size, and possibly other optional properties, such as intended contents or serving size. This establishes a database relationship connecting the user's account to the unique alphanumeric identifier and the bottle size, which is stored on a cloud server. The unique alphanumeric identifier may be reconfigured if the user wishes to identify it with another smart container or a smart container of a different size.

If the application is not installed on the user's device at the time of reading, the device will redirect the user to an installation page or link for downloading and installing application on the device, assuming the user's device has a dedicated RFID or NFC reader (e.g., a cell phone equipped with a dedicated RFID or NFC reader, such as certain models of the iPhone). Alternatively, the user may open a separate application on their device to read the passive RF communications tag in order to be directed to the link for downloading and installing an application on their device for interacting with the tag. In this instance, the user may skip this step by downloading the software specific to the container and setting up the smart container prior to interrogating the passive RF communications tag 108. If the unique alphanumeric identifier of the passive RF communications tag 108 is already associated with another smart container (i.e., one owned by another user), the software will prompt the user that it does not recognize unique alphanumeric identifier and may notify the user that the unique alphanumeric identifier is already associated with another user.

Once the database entry for the container 102 has been created, when the container 102 containing the passive RF communications tag 108 is brought in proximity (<5 cm) to a device running software specific to the container 102, the passive RF communications tag 108 can convey messages regarding the state of the container 102 in response to queries from a smart device or reader. The software records a measurement equal to the stored capacity for that container 108, and potentially other properties. For example, the software may record “24 oz of water” if the database object lists that RFID or NFC tag 108 as belonging to a 24 oz water bottle. Alternatively, it may record “2 pills of Medication X” if the database object lists that RFID or NFC tag 108 as belonging to a medication container with a dosage size of 2 pills and an intended content of Medication X.

The passive RF communications tag 108 is powered by a signal from the smartphone or reader that interrogates it. To interrogate the tag 108, the user taps a smart device against the radio frequency communication holder 103. This causes the smart device to transmit the query to the tag 108. The query powers the tag 108 and causes the tag 108 to broadcast its identifier, the size of the smart container, and/or other information stored in its microchip. The app on the smart device records the response, e.g., as an indication that the user has consumed the entire contents of the smart container. The query and response can also be triggered by bringing the user's smart device close enough to the passive RF communications tag 108 to produce a signal from the tag 108, in which case the app may confirm that the user would like to log one instance of consumption of the contents of the container. If the user has multiple smart containers, the app will register the response as being associated with the smart container assigned to the unique tag 108. Thus, the user may be able to record and monitor a variety of data across a number of smart containers to assist the user with tracking physical health and well-being.

The passive RF communications tag 108 can be used to track the amount of substance consumed from the container. For example, when the container 102 contains water, the user can track the amount of water consumed and view progress in the app. When the user taps a smart device against the radio frequency communication holder 103, the app on the smart device records the time and date associated with that response (i.e., the time and date the user consumed the number of ounces of water held in the container 102). Alternatively, the user can also manually record or adjust the time and date stamp of a particular smart container 100 record in the app. For example, if the user forgets to tap the smart device against the radio frequency communication holder 103 after consuming the contents of the container 102. The user can also adjust the amount of substance consumed. For example, if the passive RF communications tag 108 corresponds to a 24 oz water bottle but the user only consumed half of the water in the container, the user can adjust the record to indicate that only 12 oz were consumed at that date and time. The user may also delete an incorrectly recorded record (i.e., if the tag 108 was inadvertently tapped). The passive RF communications tag 108 may also communicate the location of the container 102. When the user taps a smart device against the radio frequency communication holder 103, the app on the smart device records the time and location associated with that communication. The user may view the time and location of each tap in the app on the smart device, which can be used to locate the last known location of the smart container, for example, if the user has misplaced the smart container.

The user can also assign a specific substance to a specific smart container 100. In this embodiment, when the user interrogates the passive RF communications tag 108, the amount and type of substance consumed are recorded. For example, the user may associate the tag 108 and its unique alphanumeric identifier as coffee such that when the user interrogates the tag 108, the software records that coffee, equal to the stored capacity for that container, has been consumed. Thus, the user may use multiple smart containers 100 to record consumption of a number of different substances (e.g., water, coffee, nutritional supplements).

The passive RF communications tag 108 may be tied into the electronics of the container 102. In this embodiment, the passive RF communications tag 108 can receive information from an application on the user's smart device to modify the settings of the container 102. For example, the user may set up reminders and timing in the application on the smart device instead of (or in addition to) using the additional buttons 105, 106, 107 at the bottom of the smart base 104. In this embodiment, the user uses their smart device to set the reminder settings (e.g., illumination color or timing) and the settings are conveyed to the container by taping the smart device against the radio frequency communication holder 103. For example, the user could set up an individualized sleep schedule in the application on their smart device (e.g., by modifying the length or the Day Phase or Night Phase). Additionally, the user could modify the timing or frequency of the reminders.

### Smart Band

In one embodiment, the passive RF communications tag may be contained on a smart band that can be temporarily fitted around or fixed to an ordinary bottle. The smart band may use a passive RF communications tag to communicate with a nearby smart device or dedicated RFID or NFC reader as described above. To interrogate the tag, the user taps a smart device, such as a smartphone, against the smart band. The smart band is a flexible band that can convey a variety of messages to a user. The smart band may be placed on any beverageware, container, pill bottle, or a user's wrist to convert a regular container to a smart container. The smart band allows for beverage tracking, food and/or nutrition tracking, or pill or medication tracking across a wide variety of containers. When the smart band is placed on a water bottle, the smart band can track a user's progress toward a hydration goal or prompt the user to drink from the container. When the smart band is placed on a pill container, the smart band can prompt the user to take a pill from the container or notify the user that a pill was taken from the container. When the smart band is placed on a food container, the smart band can prompt the user to consume a substance from the container and track the amount consumed from the container. The smart band may provide glowing reminders prompting the user to consume a substance from the container. Alternatively, it may provide sound or vibration reminders. The smart band may be placed on a variety of containers or moved between containers. When the smart band is placed on a new container the user can re-assign the smart band by editing the entry associating the passive RF communications tag of the smart band with the type or size of the new container.

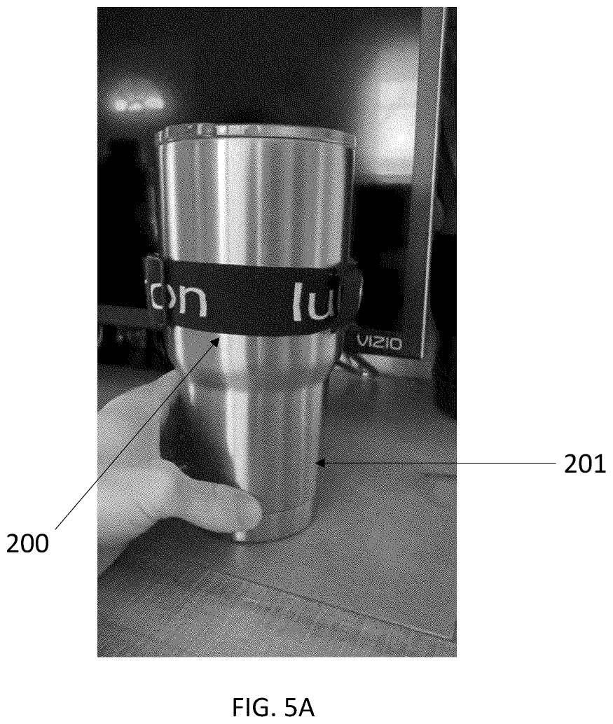

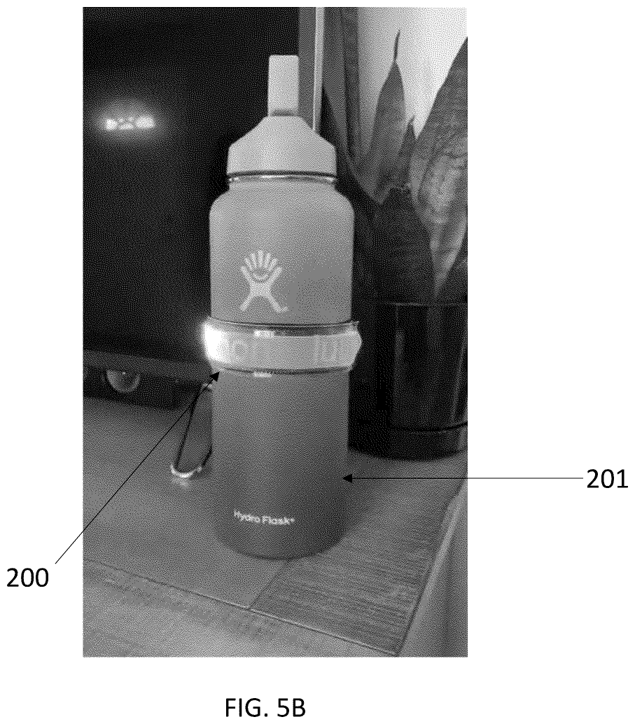

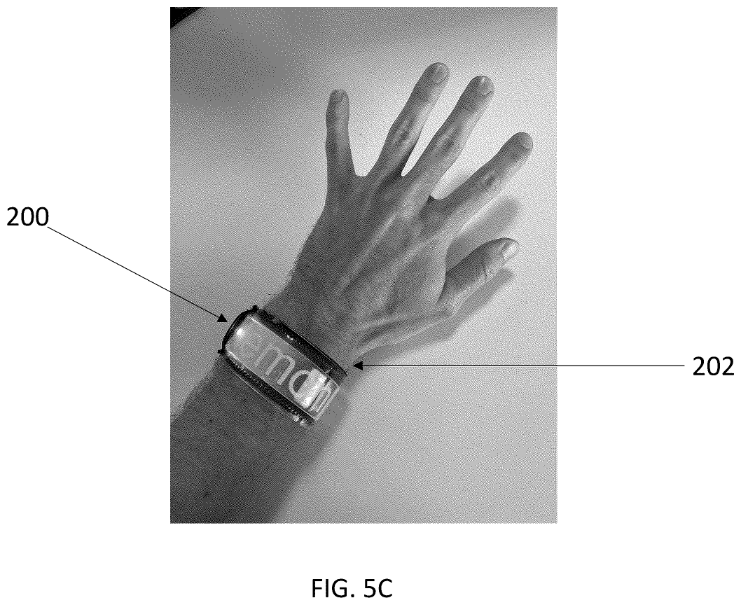

FIGS. 5A-5C show the smart band 200 on various containers. FIG. 5A shows a non-illuminated smart band 200 on a drinkware container 201. FIG. 5B shows an illuminated smart band 200 on a drinkware container 201. FIG. 5C shows the smart band 200 on a user's wrist 202. The smart band 200 may attach to the container in a variety of ways. The smart band 200 may be attached using Velcro, a belt buckle, a cinch, or any other standard forms of affixing a band.

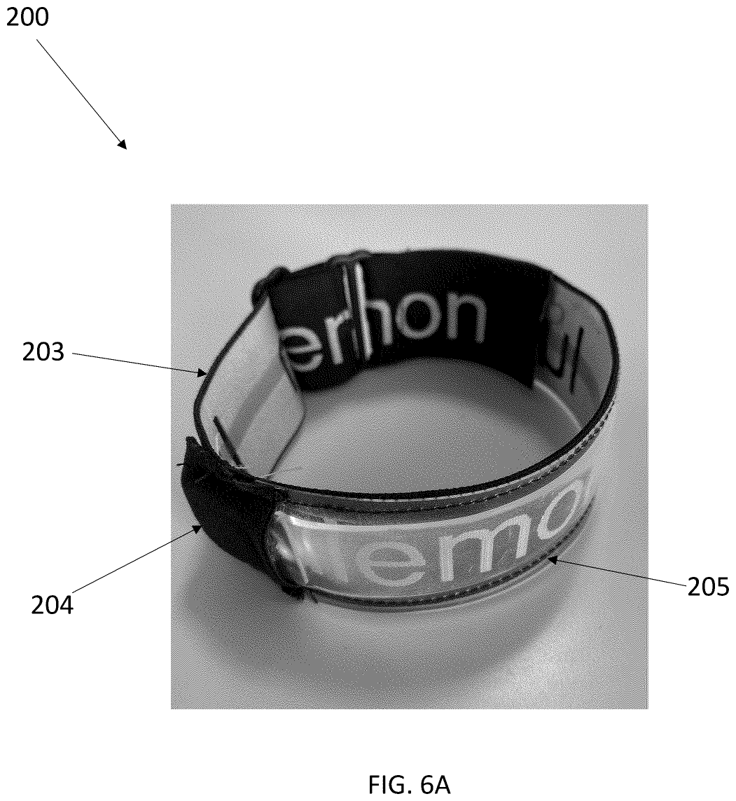

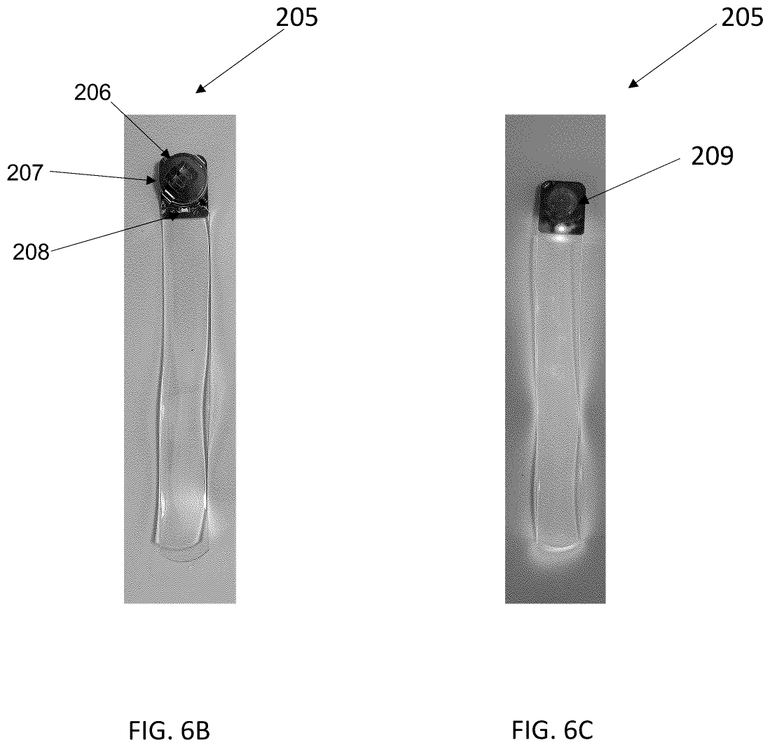

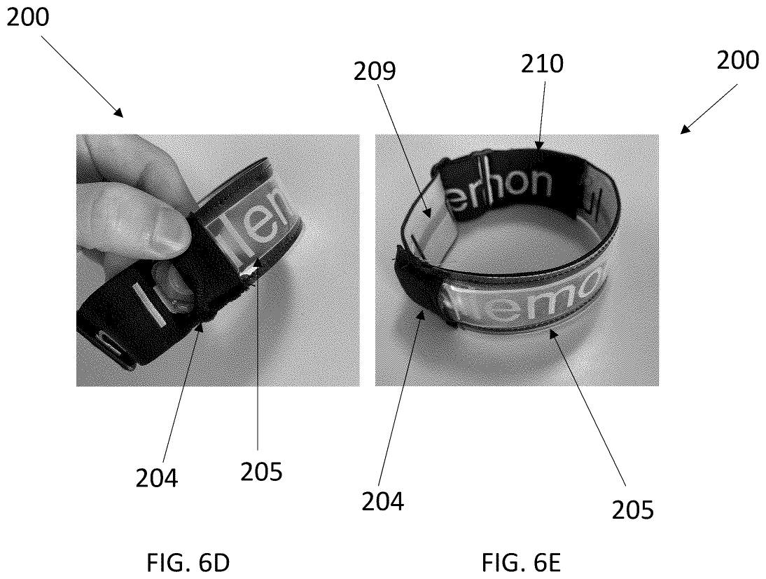

FIGS. 6A-6E show views of the smart band 200. FIG. 6A shows an assembled smart band 200. The smart band 200 may include a flexible band 203 with a fabric pocket or housing 204 and a flexible light pipe 205. The flexible band 203 may be made out of a variety of materials, including but not limited to, nylon, cotton, elastic, plastic, or other textiles and fabrics. The smart band 200 contains or holds a passive wireless communications tag, such as an RFID tag or an NFC tag, that can be interrogated using a smartphone, tag reader, or other suitable device. The tag may be located behind the fabric housing 204. Alternatively, the tag may be located anywhere on the smart band 200. The smart band 200 also includes a processor, power supply, and light(s), speaker(s), and/or actuator(s) that can be used to remind the person using the smart band 200 to ingest at least a portion of the contents of the container. The light(s), speaker(s), and/or actuator(s) may also be used to notify the person using the smart band 200 of an update to the record of the contents of the container or a celebration or reaction to the person consuming at least a portion of the contents of the container. The user may be able to program the reminders (e.g., light, audio, or haptic) using the button 209 on the smart band 200. In one embodiment, the processor and passive wireless communications tag may be operably connected so that the smart band 200 lights up after the tag has been interrogated. The light(s) of the smart band 200 may be contained in the flexible light pipe 205 as shown in FIG. 5B. Alternatively, the light(s) of the smart band 200 may be located with the other electronics (e.g., the processor and power supply).

FIGS. 6B and 6C show views of the flexible light pipe 205 without and with illumination, respectively. The flexible light pipe 205 may be contained in a portion of the flexible band 203. The flexible light pipe 205 is made up of flexible clear plastic to provide light piping. The flexible light pipe 205 is attached to a processor (e.g., on a printed circuit board) 206. The processor 206 is coupled to a battery 207 and a light source (e.g., an LED) 208. The processor 206 may also be coupled to a speaker or actuator. The speaker or actuator may be affixed to the smart band and/or flexible light pipe 205 in a similar fashion as the light source 208 (e.g., sewn, stapled, buttoned, or glued in place). The battery may be a replaceable or rechargeable battery. The top of the processor 206 contains a button 209. The button 209 controls the processor 206. The button 209 may be used to control a variety of communications depending on how long the user holds the button down or what pattern the user presses the button in. For example, the user can press the button 209 to set a glow reminder schedule, glow color, glow pattern, sound selection, start time, power, delay or snooze the next scheduled reminder, or set audio or vibrational reminders. The light pipe 205 may be removably attached to the smart band 200. Alternatively, it may be permanently attached to the smart band 200. In another embodiment, the light source (e.g., LEDs) may be extended across the smart band 200 instead of a flexible light pipe 205.

FIG. 6D shows a side view of the outside of the smart band 200 with the flexible band 203 and a fabric housing 204. The fabric housing can be pulled back to reveal the processor 206 and flexible light pipe 205. The user may be able to remove the processor 206 and flexible light pipe 205 from behind the fabric housing (e.g., to wash the smart band 200 without damaging the electronics). The fabric housing may cover the entire light pipe 205. Alternatively, the fabric housing may only cover the end of the smart band which contains the electronics (e.g., the battery 207 and processor 206). In another embodiment, the smart band 200 may not include a fabric housing 204. FIG. 6E shows a top view of the inside of the smart band 200. The inside of the smart band 200 contains a silicone grip 209 and the passive RF communications tag 210. The tag 210 is contained in a holder and the user can remove the tag 210 from the smart band 200 (e.g., to wash the smart band 200 without damaging the tag 210).

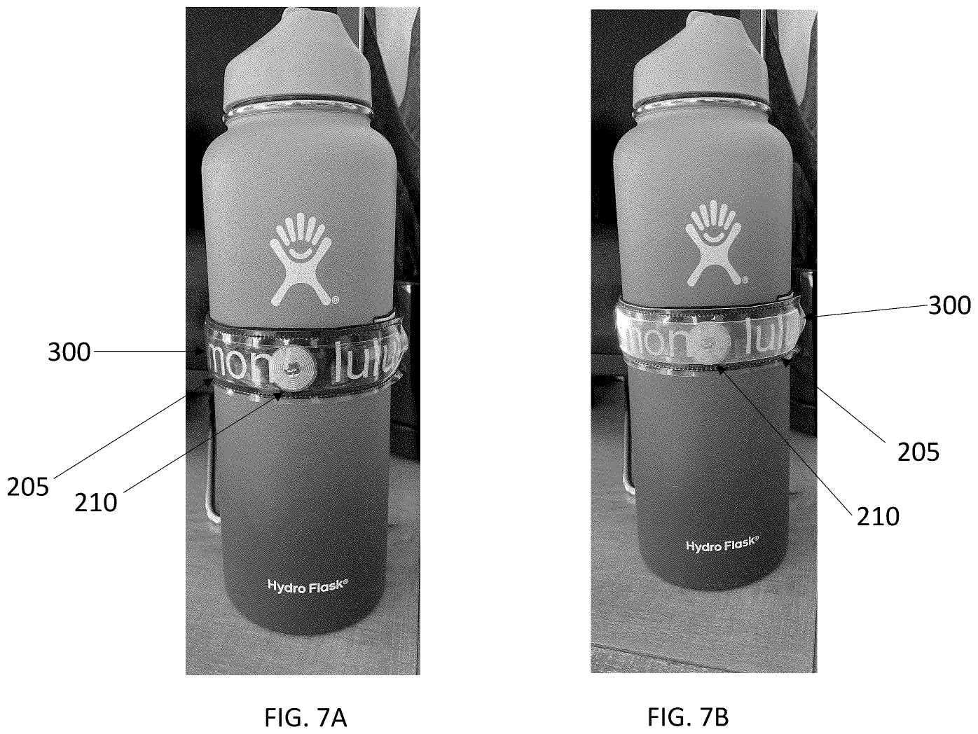

FIGS. 7A and 7B show an alternative embodiment of the smart band 300 where the passive RF communications tag 201 is contained on the flexible light pipe 205. These figures illustrate that the band can styled as desired for functional or aesthetic reasons. In addition, the tag can go anywhere on the band and isn't restricted to being on top of the electronic components.

Passive RF Communications with Various Containers

In another embodiment, the passive RF communications tag may be attached to a container via a sticker. The passive RF communication tag communicates with a nearby smart device or dedicated RFID or NFC reader as described above. To interrogate the tag, the user taps a smart device against the passive RF communications tag. The passive RF communications tag sticker may be placed on any beverageware, container, pill bottle, or a user's wrist to convert a regular container to a smart container. The sticker allows for low cost and easy implementation of beverage tracking, food and/or nutrition tracking, or pill or medication tracking across a wide variety of containers.

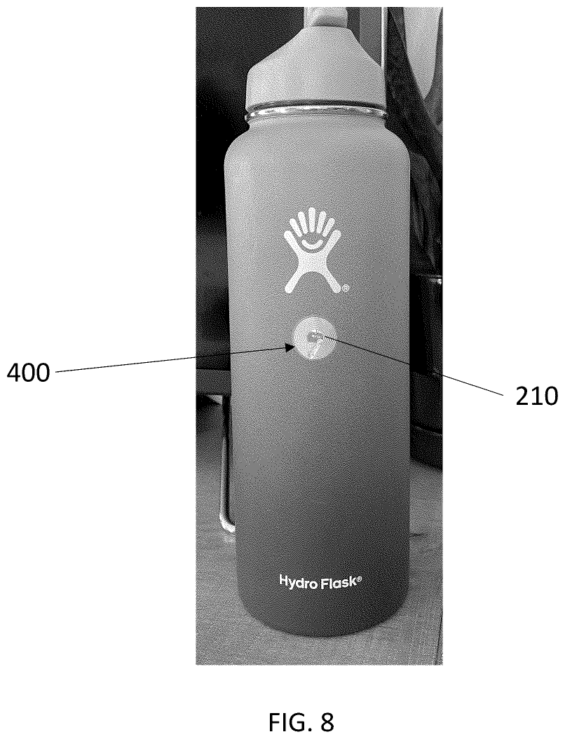

FIG. 8 shows an image of a container with a passive RF communications tag 210 attached as a sticker 400. This container (and the sticker tag 400) can be washed by hand. If desired or necessary, the sticker tag 400 can be replaced or augmented with another sticker tag 400 placed on the container.

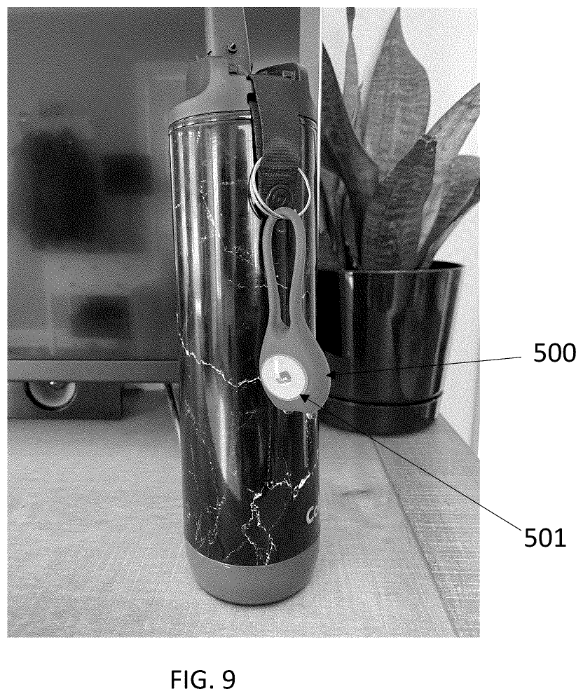

In yet another embodiment, the passive RF communications tag may be attached or contained in a dongle or keychain that attaches to a container. FIG. 9 shows an image of a dongle or keychain 500 equipped with a passive RF communications tag 501. The dongle or keychain 500 may also include a processor, power supply, and light(s), speaker(s), and/or actuator(s) that can be used to remind the person using the dongle or keychain 500 to ingest at least a portion of the contents of the container, notify the person of an update to the record of the contents of the container, or a celebration or reaction to the person consuming at least a portion of the contents of the container. The user may be able to program the reminders (e.g., light, audio, or haptic) using a button on the dongle or keychain 500.

### Smart Button

In one embodiment, a Bluetooth, Wi-Fi, and/or cellular antenna may be contained in an assembly, also called a smart button. The smart button assembly may use a Bluetooth, Wi-Fi, and/or cellular antenna to communicate with a nearby smart device, such as a user's cell phone. To communicate with the nearby smart device via the Bluetooth, Wi-Fi, and/or cellular antenna, the user presses the button of the smart button assembly. The button of the smart button assembly is operably coupled to the antenna, which then communicates to a nearby smart device. The smart button is a button that can convey a variety of messages to a user and is enclosed in a circular housing, which holds the necessary electronics. The smart button assembly is in the form of a disk and may be approximately the size of a quarter. The smart button assembly may be made of plastic or another suitable material. The smart button assembly may be partially or fully waterproof and/or water resistant.

The smart button assembly may also be contained in or attached to a removable case, allowing the smart button assembly to be moved from one object or container to another. The removable case may be functional (e.g., a means to hold the smart button) or it may also be a container itself (e.g., a keychain equipped with a small compartment to hold a substance). The removable case may cover only a portion of the smart button assembly such as the edges or the back, for example. The user may be able to switch the smart button assembly between various cases allowing for increased flexibility and mobility. The smart button assembly may be inserted in a case that is able to be directly attached to any beverageware, container, pill bottle like a sticker, for example. The smart button assembly may also be attached to any beverageware, container, pill bottle, a user's bracelet, a user's necklace, or other objects as a dongle or keychain. The smart button assembly may also be attached to an adjustable or removable band or strap that may be placed on any beverageware, container, pill bottle, or a user's wrist to convert a regular container to a smart container. The smart button assembly may be contained in a puck or smart base which is removably coupled to a container.

The smart button assembly allows for beverage tracking, food and/or nutrition tracking, or pill or medication tracking across a wide variety of containers. When the smart button assembly is placed on a water bottle, the smart button assembly can track a user's progress toward a hydration goal or prompt the user to drink from the container. When the smart button assembly is placed on a pill container, the smart button assembly can prompt the user to take a pill from the container or notify the user that a pill was taken from the container. When the smart button assembly is placed on a food container, the smart button assembly can prompt the user to consume a substance from the container and track the amount consumed from the container. The smart button assembly may provide glowing reminders prompting the user to consume a substance from the container. Alternatively, it may provide sound or vibration reminders. The smart button assembly may be placed on a variety of containers or moved between containers.

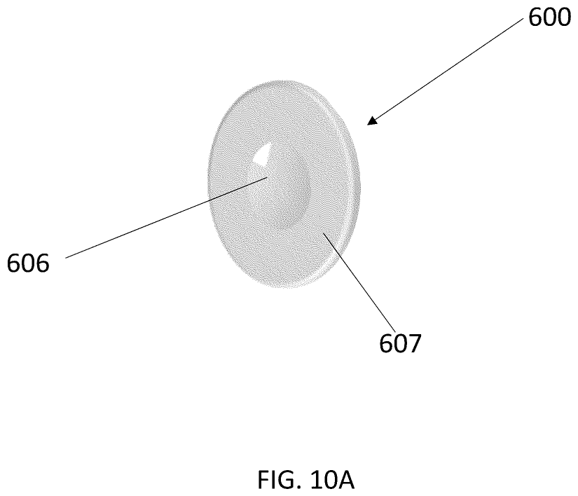

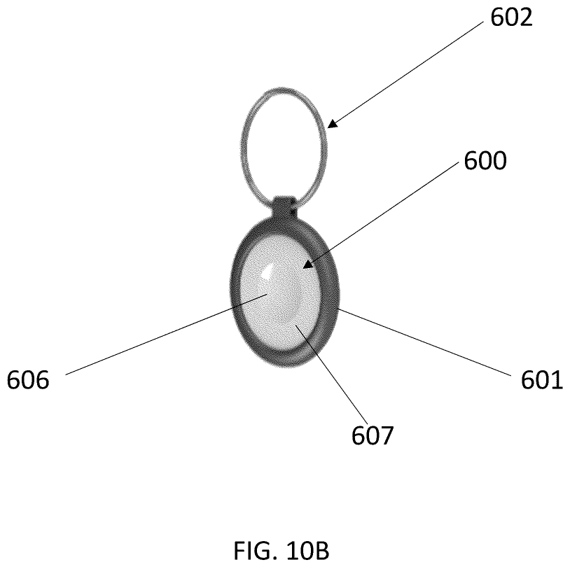

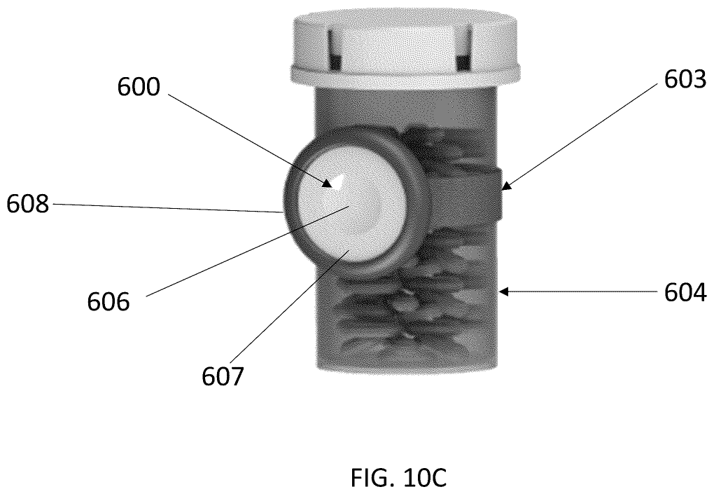

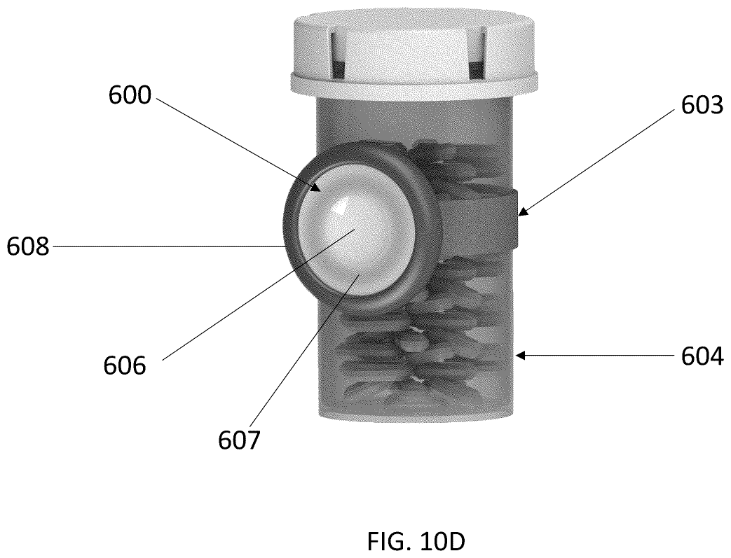

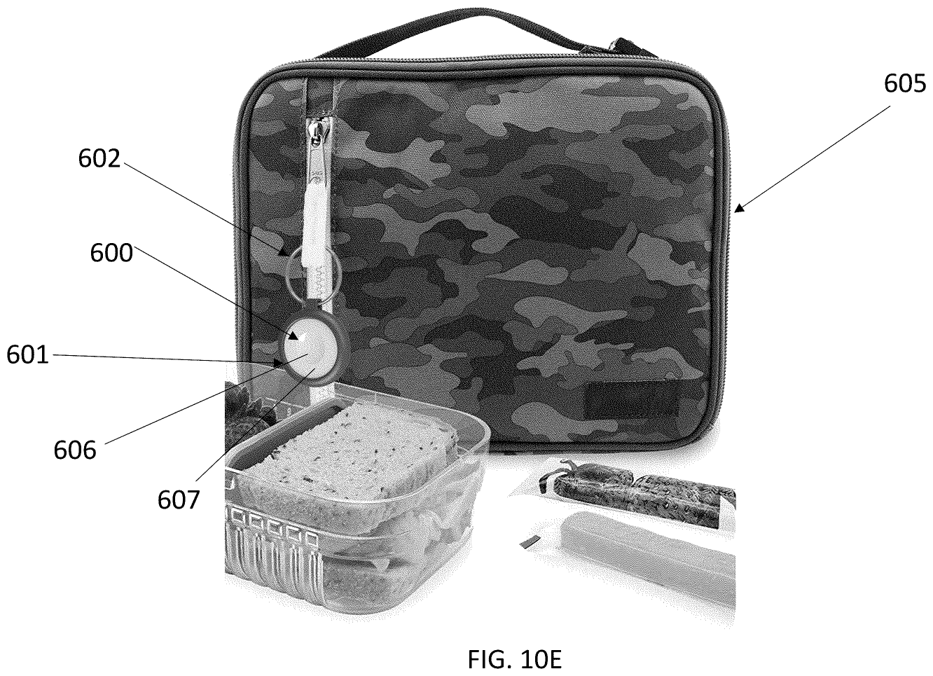

FIGS. 10A-10D show the smart button assembly by itself or contained in various cases and attached to different items. FIG. 10A shows a non-illuminated smart button assembly 600 by itself. The smart button assembly 600 includes a button 606 and housing 607 for the necessary electronics. FIG. 10B shows a non-illuminated smart button assembly 600 contained in a removable case 601 and attached to a keychain 602. FIG. 10C shows a non-illuminated smart button assembly 600 contained in a different removable case 608 and attached to a container 604 via a flexible band 603. The flexible band 603 may attach to the container in a variety of ways. For example, the flexible band 603 may be attached to the container using Velcro, a belt buckle, a cinch, or any other standard forms of affixing a band. The flexible band 603 may also be attached to a user's wrist. The flexible band 603 may be made out of a variety of materials, including but not limited to, nylon, cotton, elastic, plastic, or other textiles and fabrics. The flexible band 603 may also contain a flexible light pipe as discussed above. FIG. 10D shows an illuminated smart button assembly 600 contained in the removable case 608 and attached to a container 604 via a flexible band 603. FIG. 10E shows a non-illuminated smart button assembly 600 contained in a removable case 601 and attached to a keychain 602 which is attached to a lunchbox 605 to send food reminders, for example.

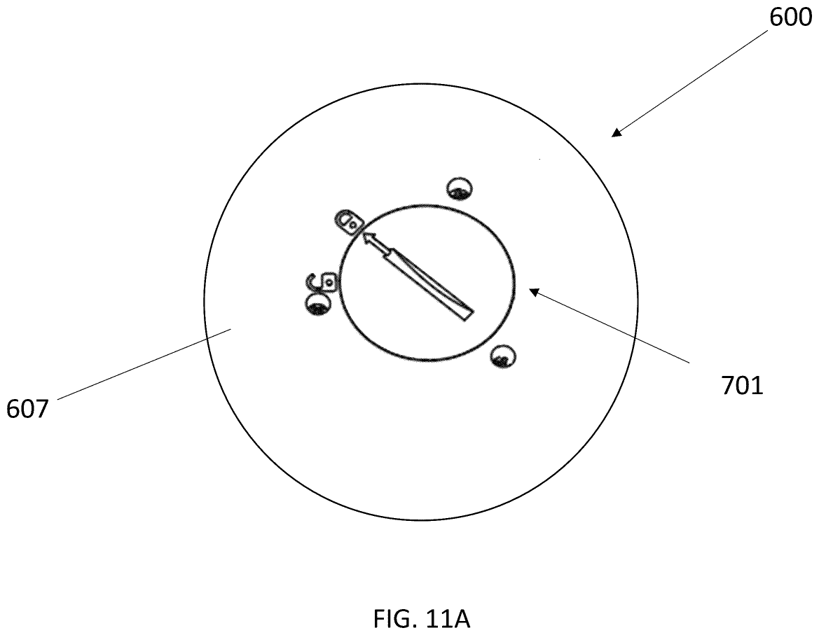

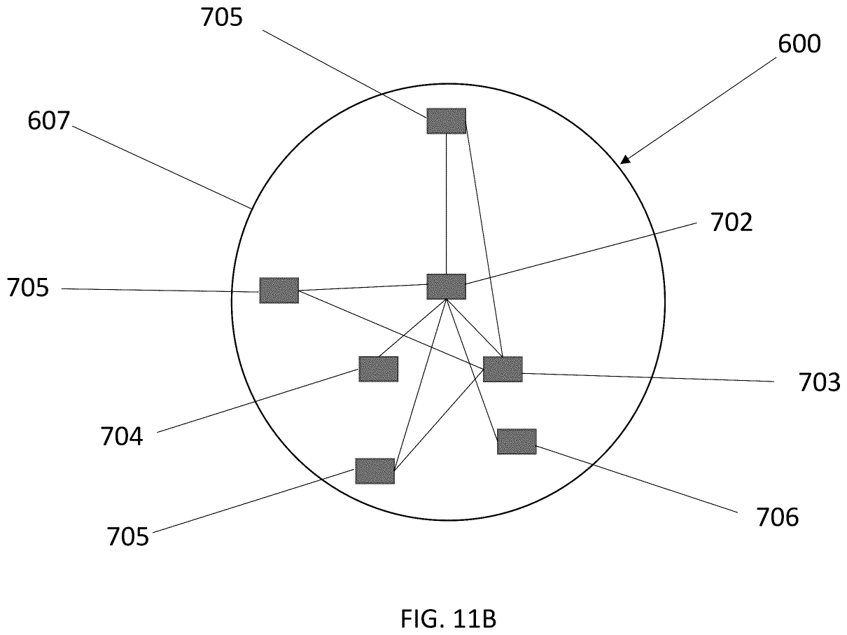

The smart button housing 607 contains or holds a Bluetooth, Wi-Fi, and/or cellular antenna. The smart button housing 607 also contains or holds a processor, power supply, and light(s), speaker(s), and/or actuator(s) that can be used to remind the person using the smart button assembly 600 to ingest at least a portion of the contents of the container. The back of the smart button housing 607 may have an electronic compartment to store the processor, power supply, and other components. The user may be able to access the battery to replace it through a door on the back of the smart button housing 607 (e.g., the battery cover 701). The battery may be accessed through a coin slot or screw, for example, as shown in FIG. 11A (described below). The light(s), speaker(s), and/or actuator(s) may also be used to notify the person using the smart button assembly 600 of an update to the record of the contents of the container or a celebration or reaction to the person consuming at least a portion of the contents of the container. In one embodiment, the processor and Bluetooth, Wi-Fi, and/or cellular antenna may be operably connected so that the smart button assembly 600 lights up, emits sound, or vibrates after the button has been pressed.

FIG. 11A shows the back of the smart button assembly 600. The back of the smart button housing 607 may have a battery cover 701, which can be unscrewed to allow the user to change the battery. FIG. 11B shows the inside of the smart button assembly 600 and the corresponding electronics. The smart button housing 607 may house various electronics, such as such as a microcontroller (MCU) or other processor 702, a battery 703, a Bluetooth, Wi-Fi, and/or cellular antenna 704, and one or more LEDs 705 as shown in FIG. 11B. The smart button assembly 600 may also include a speaker 705 and/or a vibrating motor or other actuator 706. The battery 703 is disposed in the smart button housing 607 and operably coupled to the LEDs 705 and powers the LEDs 705. The processor 702 and Bluetooth, Wi-Fi, and/or cellular antenna 704 may be operably connected so that the smart button assembly 600 lights up in response to a Bluetooth, Wi-Fi, and/or cellular signal.

The smart button assembly 600 may be used to control a variety of communications depending on how long the user holds the button down, how many times the user presses the button, or what pattern the user presses the button in. For example, the user can press the button 606 to indicate that the user has consumed a portion of the contents of the container, delay or snooze the next scheduled reminder, mark a reminder as skipped, indicate that the user has skipped consuming the contents or part of the contents of the container, or power the button on or off. For example, one press of the button 606 may indicate that the user has consumed a portion of the contents of the container attached to the smart button assembly 600. Alternatively, two presses of the button 606 may indicate that the user has snoozed the next reminder. Three presses of the button 606 may indicate that the user has skipped consuming a portion of the contents of the container. A long press of the button 606 may power on or power off the smart button assembly 600.

The smart button assembly 600 may also be equipped with a light source such as one or more light emitting diodes (LEDs) contained in the housing 607, e.g., as shown in FIG. 11B. The LEDs may be outward facing LEDs. The housing 607 may include a semi-transparent casing on the front of the smart button assembly 600 to allow light from the one or more LEDs to shine through. The semi-transparent casing may cover the entire front of the housing 607. Alternatively, the semi-transparent casing may only cover a part of the front of the housing 607 (e.g., the portion with the LEDs). The LEDs allow for a number of light patterns, chasers, flashes, pulses, back lit light patterns, color fades, or color combinations. The color, intensity, pattern, speed, or sequence of illumination from the LEDs can be customized, e.g., through an application on the user's phone. The LEDs can prompt the user to drink or consume an item from the container attached to the smart button assembly 600 (e.g., a reminder glow) or the LEDs may illuminate in response to the user pressing the button 606 (e.g., a feedback glow). The LEDs may further notify the user of specific situations, such as low battery, battery inserted, power on, power off, Bluetooth searching, Bluetooth paired, data synching, low content level, expired contents, the user's hydration level, and so on. For example, the smart button assembly 600 may pulse green once to indicate that the battery has been inserted into the smart button assembly 600. Alternatively, the smart button assembly 600 may glow green to indicate that the smart button assembly 600 has been turned on or glow red/orange to indicate that the smart button assembly 600 has been turned off. The user may be able to turn the visual reminders off or on through the app on the user's phone or smart device.

The smart button assembly 600 may also be equipped with a speaker or other noise-producing device to send audio messages to the user, e.g., as shown in FIG. 11B. The speaker may further prompt the user to consume a portion of the contents of the container attached to the smart button assembly 600 or may sound in response to the user pressing the button 606. The speaker may further communicate information regarding the progress, power state, and product state for the user, such as low battery, battery inserted, power on, power off, Bluetooth searching, Bluetooth paired, data synching, low content level, expired contents, the user's hydration level, and so on. The sound, volume, and tone of the speaker may be able to be customized, e.g., through an application on the user's phone. The user may be able to turn the audio reminders off or on through the app on the user's phone or smart device.

In some embodiments, the smart button assembly 600 may use both audio and visual notifications to notify the user. For example, if the user powers on the smart button assembly 600, the smart button assembly 600 may glow green and a chime may sound. Similarly, if the user powers off the smart button assembly 600 it may glow red/orange and another chime may sound. Alternatively, in response to the user pressing the button 606, the smart button assembly 600 may flash green three times and a different chime may sound to provide feedback to the user that the smart button assembly 600 registered the user pressing the button 606. The smart button assembly 600 may further be programed with a reminder sequence to prompt the user to consume a portion of the contents of the container. The reminder sequence may include visual, audio, and/or vibrational reminders. The reminder sequence may continue for several minutes, e.g., approximately three minutes, and repeat until the user presses the button 606 to confirm that at least a portion of the contents of the container were consumed. The user may be able to adjust the schedule and reminders through the app on the user's phone or smart device.

As discussed above, the smart button assembly 600 contains a Bluetooth, Wi-Fi, and/or cellular antenna that interacts with a user's electronic device running appropriate software (e.g., an application on the user's smart device). The smart button assembly 600 has a unique identifier or serial number. When the battery is inserted into the smart button assembly 600, it becomes discoverable to a device running appropriate software (e.g., an application on the user's smart device) via Bluetooth, Wi-Fi, and/or cellular data. If no such device exists for that user account, the software prompts the user to select the size (and in some cases other properties, such as the intended use or contents of the container) of the container associated with the smart button assembly 600. The software creates a new database entry specifying that container, its unique identifier, its size, and possibly other optional properties, such as intended contents or serving size. This establishes a database relationship connecting the user's account to the unique identifier and the container attached to the smart button assembly 600, which is stored on a cloud server. The unique identifier may be reconfigured if the user wishes to identify it with another smart container or a smart container of a different size. If the application is not installed on the user's device, the device may redirect the user to an installation page or link for downloading and installing application on the device after the user connects to the device via Bluetooth, Wi-Fi, and/or cellular data. The user may also download the software specific to the smart button assembly 600 prior to connecting to the smart button assembly 600 via Bluetooth, Wi-Fi, and/or cellular data. If the unique identifier of the smart button assembly 600 is already associated with another smart container (i.e., one owned by another user), the software may prompt the user that it does not recognize unique identifier and may notify the user that the unique identifier is already associated with another user.

Once the database entry for the container attached to the smart button assembly 600 has been created, when the user presses the button 606, the Bluetooth, Wi-Fi, and/or cellular antenna conveys messages regarding the state of the smart button assembly 600 and the app on the smart device records the response, e.g., as an indication that the user has consumed at least a portion of the contents of the container. For example, the software may record “24 oz of water” if the smart button assembly 600 is programed to belong to a 24 oz water bottle. Alternatively, it may record “2 pills of Medication X” if the smart button assembly 600 is programed belong to a medication container with a dosage size of 2 pills and an intended content of Medication X. If the user skips a reminder or dosage, the smart button assembly 600 will also record the missed response in the app.

The app may also be used by a designated third party such as a care giver or health care provider. The user would need to consent to giving the designed third party access to their data and settings of the smart button assembly 600. The designed third party may be able to set up the app and smart button assembly 600 for the user (e.g., enter the intended contents or serving size of the container associated with the smart button assembly 600). The app may also notify the designated third party with status updates (e.g., that a dosage was consumed or skipped). The designed third party may be able to access a user's data via the user's app (e.g., as an authorized user) or through a separate dashboard. The third party and/or user may also opt in to receive email, text message, or push notification alerts and reminder. The app may also export the recorded data for a given user to provide an adherence history that can be shared with a designed third party (e.g., a caregiver or health care provider). A user's data may be able to be exported as a file that can be downloaded and emailed or exported to a separate third party dashboard or database.

The user can assign a specific substance to a specific smart button assembly 600. In this embodiment, when the user presses the button 606 one time, the amount and type of substance consumed is recorded. For example, the user may associate the smart button assembly 600 as a vitamin such that when the user presses the button 606 one time, the software records that one vitamin has been consumed. Thus, the user may use multiple smart button assemblies 600 to record consumption of a number of different substances (e.g., water, coffee, nutritional supplements). Alternatively, the user may assign multiple substances to a specific smart button assembly 600. If the smart button assembly 600 is associated with multiple substances that are taken at the same time (or a similar time), the substances would be grouped in the app such that one press of the button 606 would record that all substances had been consumed. Alternatively, if the smart button assembly 600 is associated with multiple substances that are taken at different times, the timing schedule set in the app would control associating a press of the button 606 with a particular substance. For example, if Medication X is to be taken at 8 a.m. and Medication Y is to be taken at 12 p.m., when the user presses the button 606 around 8 a.m. the app will record “1 pill of Medication X.” Similarly, when the user presses the button 606 around 12 p.m., the app will record “1 pill of Medication Y.”

The user may also be able to control reminders and settings of the smart button assembly 600 through the app on their smartphone or tablet. The user may be able to color code substances they wish to track using the smart button assembly 600, adjust the glow and/or sound settings of the smart button assembly 600, view the battery level of the smart button assembly 600, and view the connectivity status of the smart button assembly 600. The app may also display a daily schedule outlining when the user should consume a specific substance associated with the smart button assembly 600. The user will also be able to view the current daily status recorded from the smart button assembly 600 such as if a notification was snoozed, a dosage was skipped, or a dosage was consumed, for example. The app may also display historical data for a period of days. The user may also be able to record symptoms and/or side effects they are experiencing when taking a particular substance through the app. For example, if the user experiences a headache or upset stomach after taking Medication X, they can record those symptoms in the app.

### Smart Button Contained in a Smart Base

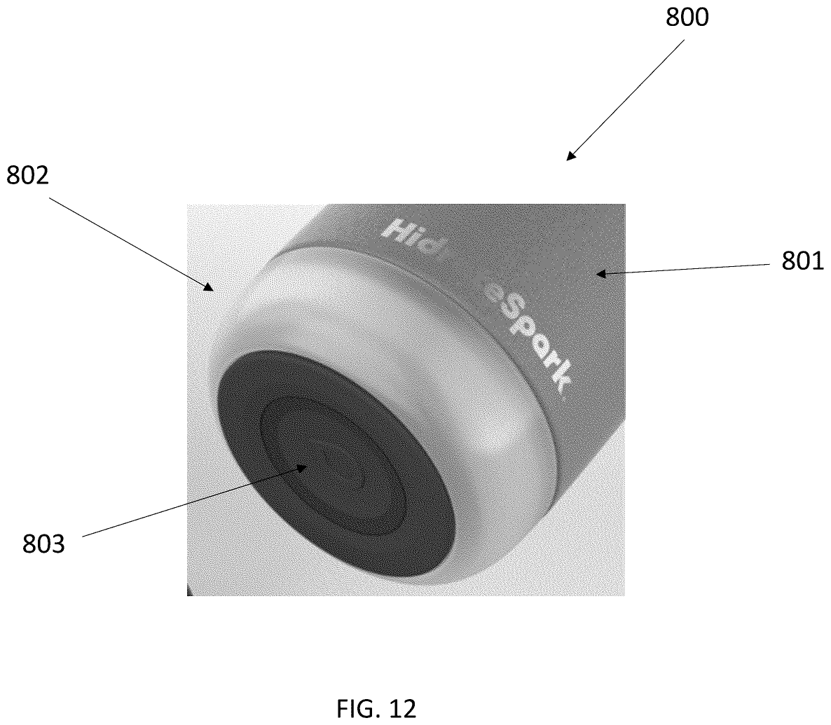

In another embodiment, the smart button may be contained in a puck or smart base which is removably coupled to a container to create a smart container. FIG. 12 shows a portion of a smart container 800, which includes a bottle or container 801 and a removable smart base 802. The smart base 802 has a button 803 on the underside of the smart base 802 as shown in FIG. 12. The button 803 operates like the button 606 described above. The button 803 may be attached to the smart base 802 as an overmold. The overmold seals the smart base 802 and protects the electronics. The button may be labeled with a symbol (as shown in FIG. 12) or words. The smart base 802 may also have LEDs inside as discussed above. The smart base 802 serves as the housing for the electronics (e.g., the processor, power supply, Bluetooth, Wi-Fi, and/or cellular antenna, and light(s), speaker(s), and/or actuator(s)) and also acts as a diffuser to diffuse the colored light from one or more LEDs over an area viewed by the user. The smart base 802 may be made out of a translucent polypropylene.

The removable smart base 802 may screw, snap, or otherwise secure to the bottom of the container 801. The smart base 802 may be attached to a container via an alignment ring as shown in FIGS. 1C-ID and may be sealed to the container 801 with a seal made of silicone or another suitable material. The container 801 may also contain a removable lid as shown in FIG. 3B, 3C, 4B, or 4C. The container 801 itself may be of any material, size, or shape suitable for holding a consumable substance. For example, the container 801 may be a water bottle, a tumbler, a pill container, a food container, a collapsible container (e.g., a collapsible food or drink container or a backpack), or another suitable container for holding a consumable substance. The container 801 may be plastic, glass, metal, silicone, and/or another suitable material. The container 801 may be a variety of sizes including, but not limited to, 2, 5, 8, 10, 12, 14, 16, 17, 18, 20, 21, 22, 24, 28, 32, 40, 64, or 128 ounces.

The button 803 may also be used to control or modify illumination reminders as described above. The container may provide reminders prompting the user to consume an item from the container or drink from the container through illumination, audio, or vibration. The user may also interact to adjust those reminders based on the button 803 at the bottom of the smart base 802. The button 803 is operably connected with the electronics contained in the smart base 802. The button 803 may be used to control a variety of communications depending on how long the user holds the button down, how many times the user presses the button, or what pattern the user presses the button in. For example, the user can press the button 803 to indicate that the user has consumed a portion of the contents of the container, delay or snooze the next scheduled reminder, mark a reminder as skipped, indicate that the user has skipped consuming the contents or part of the contents of the container, or power the smart base 802 on or off. The smart base 802 may illuminate to prompt the user to drink or consume an item from the container 801 attached to the smart base 802 or the smart base 802 may illuminate in response to the user pressing the button 803. The smart base 802 may further notify the user of specific situations, such as low battery, battery inserted, power on, power off, Bluetooth searching, Bluetooth paired, data synching, low content level, expired contents, the user's hydration level, and so on.

The smart base 802 may also be equipped with a speaker or other noise-producing device to send audio messages to the user. The speaker may further prompt the user to consume a portion of the contents of the container 801 attached to the smart base 802 or may sound in response to the user pressing the button 803. The speaker may further communicate information regarding the progress, power state, and product state for the user, such as low battery, battery inserted, power on, power off, Bluetooth searching, Bluetooth paired, data synching, low content level, expired contents, the user's hydration level, and so on. The sound, volume, and tone of the speaker may be able to be customized, e.g., through an application on the user's phone. The user may be able to turn the audio reminders off or on through the app on the user's phone or smart device. In some embodiments, the smart base 802 may use both audio and visual notifications to notify the user. The user may be able to adjust the schedule and reminders through the app on the user's phone or smart device.

As discussed above, the smart base 802 contains a Bluetooth, Wi-Fi, and/or cellular antenna that interacts with a user's electronic device running appropriate software (e.g., an application on the user's smart device). Once the database entry for the container 801 attached to the smart base 802 has been created, when the user presses the button 803, the Bluetooth, Wi-Fi, and/or cellular antenna conveys messages regarding the state of the container 801 and the app on the smart device records the response, e.g., that the user has consumed at least a portion of the contents of the container. For example, the software may record “24 oz of water” if the smart base 802 is programed to belong to a 24 oz water bottle. Alternatively, it may record “2 pills of Medication X” if the smart base 802 is programed belong to a medication container with a dosage size of 2 pills and an intended content of Medication X. If the user skips a reminder or dosage, the smart base 802 will also record the missed response in the app.

The user may also be able to control reminders and settings of the smart base 802 through the app on their smartphone or tablet. The user may be able to color code substances they wish to track using the smart container 800, adjust the glow and/or sound settings of the smart container 800, view the battery level of the smart container 800, and view the connectivity status of the smart container 800. The app may also display a daily schedule outlining when the user should consume a specific substance associated with the smart container 800. The user will also be able to view the current daily status recorded from the smart container 800 such as if a notification was snoozed, a dosage was skipped, or a dosage was consumed, for example.

### CONCLUSION

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain, using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures. Section 2111.03.

1. An assembly comprising:
a container to hold a consumable substance; and
a passive radio-frequency (RF) communication tag with a unique identifier, affixed to the container and configured to respond to a query from a smart device with the unique identifier, the unique identifier causing the smart device to update a record to indicate that a user has consumed at least a portion of the consumable substance.

2. The assembly of claim 1, wherein passive RF communication tag comprises at least one of a radio-frequency identification (RFID) tag or a near-field communication (NFC) tag.

3. The assembly of claim 1, wherein the unique identifier corresponds to an account of the user, is assigned to the container, and represents consumption of a predefined amount of the consumable substance by the user.

4. The assembly of claim 1, further comprising:
a smart base, removably coupled to the container, to hold the passive RF communication tag;
a light source, disposed in the smart base, to emit light indicating at least one of an update to the record or a prompt to the user to consume at least a portion of the consumable substance; and
a power supply, disposed in the smart base and operably coupled to the light source, to power the light source.

5. The assembly of claim 1, further comprising:
a band, disposed about the container, to hold the passive RF communication tag.

6. The assembly of claim 1, further comprising:
a dongle, removably coupled to the container, to hold the passive RF communication tag.

7. The assembly of claim 1, further comprising:
a light source, coupled to the container, to emit light indicating at least one of an update to the record, a prompt to the user to consume at least a portion of the consumable substance, or an interrogation of the passive RF communication tag.

8. (canceled)

9. (canceled)

10. (canceled)

11. (canceled)

12. (canceled)

13. (canceled)

14. (canceled)

15. A passive radio-frequency (RF) communication tag configured to be affixed to at least one of a container holding an consumable substance or a wrist of a person and store a unique identifier; and wherein the unique identifier is configured to respond to a query from a smart device, the unique identifier causing the smart device to update a record to indicate that the person has consumed at least a portion of a consumable substance from the container.

16. The passive RF communication tag of claim 15, wherein passive RF communication tag comprises at least one of a radio-frequency identification (RFID) tag or a near-field communication (NFC) tag.

17. The passive RF communication tag of claim 15, wherein the unique identifier corresponds to an account for the person, is assigned to the container, and represents consumption of a predefined amount of the consumable substance by the person.

18. A method of tracking consumption of a consumable substance stored in a container with a passive radio-frequency (RF) communication tag affixed thereto, the method comprising:
consuming, by a person, at least a portion of the consumable substance stored in the container;
interrogating the passive RF communication tag with a query from a smart device;
responding, by the passive RF communication tag, to the query with a unique identifier;
detecting, by the smart device, the unique identifier; and
updating, by the smart device, a record indicating that the person has consumed at least a portion of the consumable substance stored in the container.

19. The method of claim 18, wherein updating the record comprises:
adding at least of one a time or a location of the interrogation of the passive RF communication tag by the smart device to the record.

20. The method of claim 18, wherein updating the record comprises:
identifying a type and size of the container based on the unique identifier.

21. The method of claim 18, wherein updating the record comprises:
adding an amount of the portion of the consumable substance stored in the container consumed by the person based on the unique identifier.

22. The method of claim 21, further comprising:
adjusting, by the person, the amount of the portion of the consumable substance consumed by the person in the record.

23. The method of claim 18, further comprising:
in response to detecting the unique identifier and before updating the record, downloading, by the smart device, an application on the smart device; and
associating the unique identifier with the person, the container, and an amount of the consumable substance consumed by the person for each interrogation of the passive RF communication tag.

24. (canceled)

25. The assembly of claim 1, wherein the container comprises at least one of a liquid container, a pill container, or a food container.

26. The assembly of claim 7, wherein the light source comprises colored light-emitting diodes (LEDs).

27. (canceled)

28. (canceled)

29. The assembly of claim 1, further comprising:
a speaker affixed to the container and configured to emit sound notifications.

30. The assembly of claim 1, further comprising:
an actuator affixed to the container and configured to emit vibration patterns.
